(19) United States
(12) Patent Application Publication
(10) Pub. No.: US 2026/0201362 A1
(43) Pub. Date: Jul. 16, 2026

(54) ROBOTIC SYSTEMS AND METHODS FOR AUTONOMOUS DIRECTED EVOLUTION OF HORIZONTALLY TRANSFERRED NUCLEIC ACIDS

(71) Applicant: Massachusetts Institute of Technology, Cambridge, MA (US)

(72) Inventors: Kevin ESVELT, Cambridge, MA (US); Erika Alden DEBENEDICTIS, Cambridge, MA (US)

(73) Assignee: Massachusetts Institute of Technology, Cambridge, MA (US)

(21) Appl. No.: 19/560,490

(22) Filed: Mar. 9, 2026

Related U.S. Application Data
(62) Division of application No. 16/405,380, filed on May 7, 2019.
(60) Provisional application No. 62/668,527, filed on May 8, 2018.

(51) Int. Cl. C12N 15/10; B01L 3/00; C12M 1/00; C12M 1/26; C12M 1/32; C12M 1/36; C12Q 3/00; G01N 21/64; G01N 35/00; G01N 35/10

(52) U.S. Cl. CPC C12N 15/1058; B01L 3/502715; C12M 1/265; C12M 1/268; C12M 1/36; C12M 23/12; C12M 41/06; C12M 41/48; C12N 15/1037; C12Q 3/00; G01N 21/64; G01N 35/00693; G01N 35/0099; G01N 35/10; B01L 2300/1838; G01N 2035/00356

(57) ABSTRACT

The invention, in part, includes systems for conducting continuous directed evolution in a plurality of sample and methods of using the systems. The invention, in part also provides systems for evaluating the suitability of diverse engineered cells to accomplish directed evolution and methods of use of such systems.

### RELATED APPLICATIONS

This application claims benefit under 35 U.S.C. § 119(e) of U.S. Provisional application No. 62/668,527 filed May 8, 2018, the entire contents of which is incorporated by reference herein.

### FIELD OF THE INVENTION

This disclosure relates generally to systems, components, and methods for conducting continuous directed evolution simultaneously in a plurality of samples.

### BACKGROUND OF THE INVENTION

Laboratory evolution has generated many biomolecules, e.g., nucleic acids and proteins, with desired properties. However, conventional laboratory evolution strategies rely on discrete mutation-selection rounds, and a single round of directed evolution typically requires days with frequent intervention by a researcher, making complex evolution processes impractical to perform in the laboratory. Scientists have developed a platform that enables the continuous directed evolution of gene-encoded molecules that can be linked to protein production in a host cell, for example, in an E. coli cell. Scientists further developed a method of evolving a gene of interest by linking an activity of a molecule encoded by the gene of interest to the transfer of the gene of interest from cell to cell.

More recently, scientists have developed systems and methods for conducting Phage-Assisted Continuous Evolution (PACE) of a protein; see for example U.S. Pat. Nos. 9,394,537 and 9,771,574. The PACE method includes providing a flow of host cells, for example, a flow of bacterial host cells through container, and a population of phage vectors encoding a gene of interest replicating in the flow of host cells under conditions allowing for replication and mutation of the phage genomes. The host cells may include a gene required for the generation of infectious phage under the control of a conditional promoter that is activated by a desired function of the gene of interest, thus conferring a selective advantage to phage genomes acquiring a gain-of-function with the gene of interest. The PACE method includes contacting a population of bacterial host cells with a population of phage but is limited by the low number and type of interactions that can be performed in parallel.

### SUMMARY OF THE INVENTION

In one aspect of the invention, a system for conducting discretized continuous directed evolution simultaneously in a plurality of samples is provided, the system including: a plurality of receptacles, each receptacle configured to contain a sample comprising cells and for liquid exchange, wherein one or more of the receptacles includes one or more horizontally transferable nucleic acids; a liquid handling robot configured to perform liquid exchange in one or more receptacles in the plurality of receptacles to facilitate a mixing of the samples; and a sensor configured to detect at least one detectable parameter of one or more of the samples. In some embodiments, at least one of the cells does not include a horizontally transferable nucleic acid. In some embodiments, one or more of the horizontally transferable nucleic acids undergo directed evolution. In certain embodiments, the mixing is within a sample. In some embodiments, the horizontally transferable nucleic acids are bacteriophages, including filamentous phages such as M13/f1/fd, temperate phages such as lambda, or lytic phages such as T1-T7. In some embodiments, the horizontally transferable nucleic acids are conjugative plasmids, such as F, F0lac, Col1b, or RP4/RK2. In some embodiments, the horizontally transferable nucleic acids are secreted by at least a portion of the cells in one or more of the samples in the plurality of receptacles, wherein the means of secreting includes a DNA export system. In certain embodiments, the DNA export system includes a N. gonorrhoeae DNA export system. In some embodiments, the secreted nucleic acids are internalized by at least a portion of the cells in one or more of the samples, wherein the internalization means includes a DNA transporter or other bacterial competence machinery. In some embodiments, the horizontally transferable nucleic acids encode one or more genes that produce one or more biomolecules that exhibit at least one direct sensor-detectable parameter. In certain embodiments, one or more of the cells produce an indirect sensor-detectable parameter when triggered by the production of one or more biomolecules encoded by the horizontally transferable nucleic acids. In some embodiments, the system also includes a feedback controller configured to make at least one real-time adjustment in one or more samples in the plurality of receptacles based upon the at least one detectable parameter. In some embodiments, the sensor is configured to detect at least one detectable parameter of one or more samples while the one or more samples are in the plurality of receptacles. In certain embodiments, the detection of the detectable parameter determines a characteristic of one or more of the samples in the plurality of receptacles. In some embodiments, one or more of the samples in the plurality of receptacles comprise a population of bacteriophage. In certain embodiments, the determined characteristic includes at least one of: selection, positive selection, negative selection, an enzyme activity, phage population size, bacterial population size, gene transcription, and biomolecule evolution in one or more of the samples in the plurality of receptacles. In some embodiments, the detecting includes detecting one of more of a presence, an absence, and an amount of the detectable parameter. In some embodiments, the detectable parameter includes one or more of absorbance, luminescence, and fluorescence. In some embodiments, the sensor is configured to detect luminescence in one or more of the samples in the plurality of receptacles. In certain embodiments, the sensor is configured to detect fluorescence in one or more of the samples in the plurality of receptacles. In some embodiments, the sensor is configured to detect one or more of: absorbance, luminescence, and fluorescence in one or more of the samples in the plurality of receptacles. In some embodiments, the sensor is constructed and arranged as an integrated plate reader configured to determine at least one detectable parameter of one or more of the samples in the plurality of receptacles. In certain embodiments, the one or more of the samples comprise a population of bacteriophage and the determined characteristic includes a level of one or both of pIII and pIII* transcription within one or more of the samples in the plurality of receptacles. In some embodiments, the sensor is configured to detect over a time period of at least 1, 3, 6, 12, 18, 24, 36, 48, or more hours. In some embodiments, the sensor is configured to detect continuously over the time period. In certain embodiments, the sensor is configured to detect once every 1, 5, 10, 15, 20, 30, 60, 120, or more minutes. In some embodiments, the sensor is configured to detect at a regular interval over 1, 3, 6, 12, 18, 24, 36, 48, or more hours. In some embodiments, the real-time adjustment includes adjusting a condition in one or more of the samples in the plurality of receptacles. In some embodiments, the real-time adjustment includes modifying at least one of a: (a) fluid exchange frequency, (b) fluid exchange volume, (c) sample composition, (d) selection parameter, (f) selection stringency, and (g) selection goal in at least one of the samples in the plurality of receptacles. In certain embodiments, one or more of the samples in the plurality of receptacles comprise a population of bacteriophage and the real-time adjustment includes at least one of a: (a) fluid exchange frequency, (b) fluid exchange volume, (c) sample composition, (d) selection parameter, (f) selection stringency, and (g) selection goal in at least one of the bacteriophage-containing samples. In some embodiments, one or more of the samples in the plurality of receptacles comprise a population of bacteriophage, and the feedback controller is configured to adjust a positive selection strength of at least one of the bacteriophage-containing samples in response to the at least one detectable parameter. In certain embodiments, one or more of the samples in the plurality of receptacles comprise a population of bacteriophage, and the feedback controller is configured to adjust a negative selection strength of at least one bacteriophage-containing sample in response to the at least one detectable parameter. In some embodiments, one or more of the samples in the plurality of receptacles comprise a population of bacteriophage, and the feedback controller is configured to adjust a bacteriophage mutation rate in at least one bacteriophage-containing sample in response to the at least one detectable parameter. In some embodiments, one or more of the samples in the plurality of receptacles comprise a population of bacteriophage, and the feedback controller is configured to adjust a selection goal of at least one bacteriophage-containing sample in response to the at least one detectable parameter. In some embodiments, the sensor is configured to detect at least a first detectable parameter and a second detectable parameter in one or more of the samples in the plurality of receptacles; and wherein the feedback controller is configured to make one or more real-time adjustments in the one or more of the samples in the plurality of receptacles based at least in part on the first detectable parameter and the second detectable parameter. In certain embodiments, the means of detecting the first and second detectable parameters includes detecting a first and a second detectable agent, respectively. In some embodiments, the first detectable agent includes a first fluorescent molecule and the second detectable agent comprise a second fluorescent molecule. In some embodiments, one or more of the samples in the plurality of receptacles comprise a population of bacteriophage. In some embodiments, one or more of the samples in the plurality of receptacles comprise a polymerase. In certain embodiments, the polymerase includes a T7 RNA polymerase. In some embodiments, the first detectable agent includes an mCherry molecule and its presence in the sample indicates the first characteristic of positive selection in the sample and the second detectable agent includes a GFP molecule and its presence in the indicates the second characteristic of negative selection in the sample; and wherein the feedback controller is configured to adjust one or more of the samples in the plurality of receptacles in real time, based at least in part on the determination of at least one of the first characteristic and the second characteristic. In some embodiments, the first detectable agent includes a first luminescent molecule and the second detectable agent includes a second luminescent molecule. In certain embodiments, the feedback controller is configured to adjust one or more of the samples in the plurality of receptacles in real time by adding an inducing agent to one or more of the samples in the plurality of receptacles. In some embodiments, the inducing agent includes at least one of L-arabinose, anhydrotetracycline, theophylline, 2,4-diacetylphophloroglucinol, cuminic acid, 3-oxohexanoyl-homoserine lactone, vanillic acid, isopropyl β-D-1-thiogalactopyranoside (IPTG), choline chloride, naringenin, 3,4-dihydroxybenzoic acid, sodium salicylate, and 3-hydroxytetradecanoyl-homoserine lactone. In some embodiments, the system also includes at least one temperature element configured to selectively modulate a temperature of one or more of the samples in the plurality of receptacles. In some embodiments, the feedback controller is configured to activate the temperature element in real-time based at least in part on a temperature of one or more of the samples in the plurality of receptacles. In certain embodiments, the feedback controller is configured to activate the temperature element to modulate the temperature in real-time to hold at 37° C. In some embodiments, the feedback controller is configured to activate the temperature element to modulate the temperature in real time to a temperature between one or more of: 25° C. and 40° C., 30° C. and 40° C., 32° C. and 38° C., 34° C. and 37° C., and 34.5° C. and 37.5° C. In some embodiments, the liquid handling robot further includes one or more reservoirs, wherein each reservoir is configured to hold an independently selected liquid exchange fluid. In certain embodiments, the independently selected liquid exchange fluid includes a bacteriophage solution. In some embodiments, the liquid handling robot is configured to selectively dispense the bacteriophage solution into one or more receptacles in the plurality of receptacles. In some embodiments, the independently selected liquid exchange fluid includes an inducing agent. In some embodiments, the reservoir has at least four ports including a first port to fluidly couple the reservoir to a water line, a second port to fluidly couple the reservoir to a cleaning fluid line, a third port to fluidly couple the reservoir to a liquid exchange fluid line, and a fourth port to fluidly couple the reservoir to a drain line. In certain embodiments, the liquid handling robot further includes one or more peristaltic pumps configured to selectively perform liquid exchange in one or more of the samples in the plurality of receptacles. In some embodiments, each sample in the plurality of receptacles is contained in a separate receptacle. In some embodiments, the system includes one or more sets of receptacles, wherein a set includes at least 1, 5, 10, 15, 20, 30, 40, 50, 60, 70, 80, 90, 100, 150, 200, 250, 300, 350, 400, 450, 500, 550, 600, 650, 700, 750, 800, 850, 900, 950, 1000 or more receptacles. In certain embodiments, the system includes at least 1, 2, 3, 4, 5, 6, 7, 8, 9, 10, 15, 20, 25, or more sets of receptacles, each with an independently selected number of receptacles. In some embodiments, one or more of the sets of receptacles includes 96, 192, or 384 receptacles. In some embodiments, the sensor is configured to detect at least one parameter of all of the samples within the one or more sets of receptacles. In certain embodiments, the feedback controller is configured to make at least one real-time adjustment in all of the samples in the plurality of receptacles based upon the at least one detectable parameter. In some embodiments, the detecting includes transferring an aliquot of each of the one or more samples in the plurality of receptacles into a separate detection receptacle, and wherein the sensor is configured to detect at least one parameter of the transferred aliquot while the transferred aliquot is in the detection receptacle. In some embodiments, the one or more samples includes at least two samples. In some embodiments, the one or more samples includes a plurality of samples. In certain embodiments, the one or more cells includes at least two cells. In some embodiments, the one more cells includes a plurality of cells. In some embodiments, the one or more horizontally transferable nucleic acids includes 2, 3, 4, 5, 6 or more independently selected horizontally transferable nucleic acids.

According to another aspect of the invention, methods for conducting discretized continuous directed evolution simultaneously in a plurality of samples are provided, the methods including: providing one or more receptacle in a plurality of receptacles, with one or more samples, wherein (i) each sample is in a separate receptacle in the plurality of receptacles; (ii) one or more independently selected receptacles in the plurality of receptacles includes a cell; and (iii) one or more independently selected receptacles in the plurality of receptacles includes one or more horizontally transferable nucleic acids; providing a liquid handling robot configured to perform liquid exchange in one or more receptacles in the plurality of receptacles; facilitating mixing in one or more receptacles in the plurality of receptacles; detecting at least a first detectable parameter in one or more of the samples in the plurality of samples; and modifying one or more of the samples in the plurality of receptacles in real-time based at least in part on the first detectable parameter of the one or more of the samples. In certain embodiments, at least one of the horizontally transferable nucleic acids is undergoing directed evolution. In some embodiments, one or more cells in the one or more samples does not comprise a horizontally transferable nucleic acid. In some embodiments, at least one of the one or more cells that does not include the horizontally transferable nucleic acid is undergoing directed evolution. In some embodiments, the mixing is within the sample. In certain embodiments, the facilitated mixing includes mixing (a) one or more of the cells that does not include a horizontally transferable nucleic acid undergoing directed evolution and (b) one or more of the cells including a horizontally transferable nucleic acid undergoing directed evolution. In some embodiments, the horizontally transferable nucleic acids are bacteriophages, including filamentous phages such as M13/f1/fd, temperate phages such as lambda, or lytic phages such as T1-T7. In some embodiments, the horizontally transferable nucleic acids are conjugative plasmids, such as F, F0lac, Col1b, or RP4/RK2. In certain embodiments, the horizontally transferable nucleic acids are secreted by at least a portion of the cells in the plurality of receptacles, wherein the means of secreting includes a DNA export system. In some embodiments, the DNA export system includes a N. gonorrhoeae DNA export system. In some embodiments, the secreted nucleic acids are internalized by at least a portion of the cells in the plurality of receptacles, wherein the internalization means includes a DNA transporter or other bacterial competence machinery. In some embodiments, the horizontally transferable nucleic acids encode one or more genes that produce one or more biomolecules that exhibit at least one direct sensor-detectable parameter. In certain embodiments, one or more of the cells produce an indirect sensor-detectable parameter when triggered by the production of one or more biomolecules encoded by the horizontally transferable nucleic acids. In some embodiments, a feedback controller is also provided and is configured to make at least one real-time adjustment in one or more of the samples in the plurality of receptacles based upon the first detectable parameter. In some embodiments, a sensor is also provided and is configured to detect at least the first detectable parameter of one or more of the samples while the one or more samples are in the plurality of receptacles. In certain embodiments, the detection of the first detectable parameter determines a characteristic of one or more of the samples in the plurality of receptacles. In some embodiments, one or more of the samples in the plurality of receptacles comprise a population of bacteriophage. In some embodiments, the determined characteristic includes at least one of: selection, positive selection, negative selection, an enzyme activity, phage population size, bacterial population size, gene transcription, and biomolecule evolution in one or more of the samples in the plurality of receptacles. In some embodiments, the detecting includes detecting one of more of a presence, an absence, and an amount of the first detectable parameter. In certain embodiments, the method also includes determining a characteristic of one or more of the samples, based at least in part on the first detectable parameter. In some embodiments, the determined characteristic includes at least one of:
selection, positive selection, negative selection, an enzyme activity, phage population size, gene transcription, biomolecule evolution, and promoter activity in the one or more of the samples. In some embodiments, the detecting includes detecting one of more of a presence, an absence, and an amount of the first detectable parameter. In some embodiments, the first detectable parameter includes one of more of absorbance, luminescence, and fluorescence. In certain embodiments, one or more samples in the plurality of samples are modified in real-time based upon at least one of the detected absorbance, luminescence, and fluorescence. In some embodiments, one or more of the samples comprise a bacteriophage population, and wherein the modification of one or more samples in the plurality of receptacles is selected for one or more of: (a) modifying a positive selection strength of at least one sample comprising a bacteriophage population in response to the first detectable parameter; (b) modifying a negative selection strength of at least one sample comprising a bacteriophage population in response to the first detectable parameter; (c) modifying a mutation rate of at least one sample comprising a bacteriophage population in response to the first detectable parameter; and (d) modifying a selection goal of at least one sample comprising a bacteriophage population in response to the first detectable parameter. In some embodiments, modifying one or more of the samples in the plurality of receptacles includes adding an inducing agent to one or more of the samples in the plurality of receptacles in response to the first detectable parameter. In certain embodiments, the method also includes detecting a second parameter in one or more of the samples in the plurality of receptacles; and modifying one or more samples in the plurality of receptacles in real-time based at least in part on the second detected parameter of the one or more of the samples. In some embodiments, the method also includes: providing one or more reservoirs, wherein each reservoir is configured to hold and selectively dispense an independently selected liquid exchange fluid; sterilizing one or more of the reservoirs with a solution comprising bleach; and placing one or more of the independently selected liquid exchange fluids into a selected reservoir of the one or more reservoirs. In some embodiments, at least one of the independently selected liquid exchange fluids includes a bacterial culture. In certain embodiments, at least one of the independently selected liquid exchange fluids includes a bacteriophage solution. In some embodiments, at least one of the independently selected liquid exchange fluids includes an inducing agent. In some embodiments, at least one of the one or more reservoirs has at least four ports including a first port to fluidly couple the reservoir to a water line, a second port to fluidly couple the reservoir to a cleaning fluid line, a third port to fluidly couple the reservoir to a liquid exchange fluid line, and a fourth port to fluidly couple the reservoir to a drain line. In certain embodiments, the liquid handling robot further includes one or more peristaltic pumps configured to selectively perform liquid exchange in one or more samples in the plurality of receptacles. In some embodiments, each sample is in a separate receptacle and the method includes a plurality of samples each in a separate receptacle. In some embodiments, the sterilizing step includes: washing the reservoir with a bleach solution comprising at least 8%, 9%, 10%, 11%, or 12% bleach; and rinsing the reservoir with a solution comprising water. In some embodiments, the bleach solution includes 10% bleach. In certain embodiments, the rinsing step is performed at least 2, 3, 4 5, 6, 7, 8, 9, 10 or more times. In some embodiments, the detecting step includes transferring an aliquot of one of the samples from the receptacle containing the sample into a detection receptacle, and detecting the at least one parameter in the aliquoted sample while the aliquot is in the detection receptacle. In some embodiments, the modifying step includes modifying one or more of the samples within the receptacles in real-time based upon the first detectable parameter. In some embodiments, the method includes use of any embodiment of an aforementioned system. In certain embodiments, one or more samples includes at least two samples. In some embodiments, one or more samples includes a plurality of samples. In some embodiments, one or more cells includes at least two cells. In certain embodiments, one or more cells includes a plurality of cells. In some embodiments, the one or more horizontally transferable nucleic acids includes 2, 3, 4, 5, 6 or more independently selected horizontally transferable nucleic acids.

According to yet another aspect of the invention, a system for evaluating the suitability of diverse engineered cells to accomplish directed evolution is provided, the system including: a plurality of receptacles, each receptacle configured to contain a sample comprising cells and for liquid exchange, wherein one or more of the receptacles includes one or more horizontally transferable nucleic acids; a liquid handling robot configured to perform liquid exchange in one or more receptacles in the plurality of receptacles to facilitate the mixing of the samples; and a sensor configured to detect at least one first detectable parameter of one or more of the samples, wherein: at least one of the horizontally transferable nucleic acids is preselected to be evolved; one or more of the samples in the plurality of the receptacles includes one or more of the horizontally transferable nucleic acids preselected to be evolved; one or more of the samples in the plurality of receptacles does not include the one or more horizontally transferable nucleic acids preselected to be evolved; at least one cell in one or more of the samples in the plurality of receptacles is an engineered cell encoding one or more independently selected engineered alterations capable of determining an activity of at least one of the horizontally transferable nucleic acids preselected to be evolved; and wherein detecting the at least one first detectable parameter includes detecting the activity determined by the one or more independently selected engineered alterations. In certain embodiments, the liquid handling robot is configured to perform liquid exchange in one or more of the receptacles comprising the samples and to facilitate mixing of the samples comprising the horizontally transferable nucleic acids preselected to be evolved with the samples that do not comprise the horizontally transferable nucleic acids preselected to be evolved. In some embodiments, the sensor is configured to detect at least one detectable parameter of one or more of the samples, wherein the first detectable parameter includes the determined activity of the horizontally transferable nucleic acid preselected to be evolved. In some embodiments, the first detectable parameter includes the activity of the horizontally transferrable nucleic acid preselected to be evolved, and detecting the first detectable parameter includes determining in one or more sample in the plurality of receptacles, the activity of the horizontally transferable nucleic acid preselected to be evolved. In certain embodiments, the mixing is within the sample. In some embodiments, the horizontally transferable nucleic acids are bacteriophages, including filamentous phages such as M13/f1/fd, temperate phages such as lambda, or lytic phages such as T1-T7. In some embodiments, the horizontally transferable nucleic acids are conjugative plasmids, such as F, F0lac, Col1b, or RP4/RK2. In certain embodiments, the horizontally transferable nucleic acids are secreted by at least a portion of the cells in one or more of the samples in the plurality of receptacles, wherein the means of secreting includes a DNA export system. In some embodiments, the DNA export system includes a N. gonorrhoeae DNA export system. In some embodiments, the secreted nucleic acids are internalized by at least a portion of the cells in the one or more of the samples in the plurality of receptacles, wherein the internalization means that includes a DNA transporter or other bacterial competence machinery. In some embodiments, the horizontally transferable nucleic acids encode one or more genes that produce one or more biomolecules that exhibit at least one direct sensor-detectable parameter. In certain embodiments, one or more of the cells produce an indirect sensor-detectable parameter when triggered by the production of one or more biomolecules encoded by the horizontally transferable nucleic acids. In some embodiments, the system also includes a feedback controller configured to make at least one real-time adjustment in one or more of the samples in the plurality of receptacles based upon the at least one detectable parameter. In some embodiments, the sensor is configured to detect at least one detectable parameter of one or more of the samples while the one or more samples are in the receptacles. In certain embodiments, the detection of the detectable parameter determines a characteristic of one or more of the samples in the plurality of receptacles. In some embodiments, one or more of the samples in the plurality of receptacles comprise a population of bacteriophage. In some embodiments, the determined characteristic includes at least one of: selection, positive selection, negative selection, an enzyme activity, phage population size, bacterial population size, gene transcription, and biomolecule evolution in one or more of the samples in the plurality of receptacles. In some embodiments, the detecting includes detecting one of more of a presence, an absence, and an amount of the detectable parameter. In certain embodiments, the detectable parameter includes one or more of absorbance, luminescence, and fluorescence. In some embodiments, the sensor is configured to detect luminescence in one or more of the samples in the plurality of receptacles. In some embodiments, the sensor is configured to detect fluorescence in one or more of the samples in the plurality of receptacles. In some embodiments, the sensor is configured to detect one or more of: absorbance, luminescence, and fluorescence in one or more of the samples in the plurality of receptors. In some embodiments, the sensor is constructed and arranged as an integrated plate reader configured to determine at least one detectable parameter of one or more of the samples in the plurality of receptacles. In certain embodiments, the one or more samples in the plurality of samples includes a population of bacteriophage and the determined characteristic includes a level of one or both of pIII and pIII* transcription within one or more of the samples in the plurality of receptacles. In some embodiments, the sensor is configured to detect over a time period of at least 1, 3, 6, 12, 18, 24, 36, 48, or more hours. In some embodiments, the sensor is configured to detect continuously over the time period. In certain embodiments, the sensor is configured to detect once every 1, 5, 10, 15, 20, 30, 60, 120, or more minutes. In some embodiments, the sensor is configured to detect at a regular interval over 1, 3, 6, 12, 18, 24, 36, 48, or more hours. In some embodiments, the real-time adjustment includes adjusting a condition in one or more of the samples in the plurality of receptacles. In some embodiments, the real-time adjustment includes modifying at least one of a: (a) fluid exchange frequency, (b) fluid exchange volume, (c) sample composition, (d) selection parameter, (f) selection stringency, and (g) selection goal in one or more of the samples in the plurality of receptacles. In certain embodiments, one or more of the samples in the plurality of receptacles includes a population of bacteriophage and the real-time adjustment includes at least one of a: (a) fluid exchange frequency, (b) fluid exchange volume, (c) sample composition, (d) selection parameter, (f) selection stringency, and (g) selection goal in at least one of the bacteriophage-containing samples. In some embodiments, one or more of the samples in the plurality of receptacles includes a population of bacteriophage, and the feedback controller is configured to adjust a positive selection strength of one or more of the bacteriophage-containing samples in response to the at least one first detectable parameter. In some embodiments, one or more of the samples in the plurality of receptacles includes a population of bacteriophage, and the feedback controller is configured to adjust a negative selection strength of one or more of the bacteriophage-containing samples in response to the at least one first detectable parameter. In certain embodiments, one or more of the samples in the plurality of receptacles includes a population of bacteriophage, and the feedback controller is configured to adjust a bacteriophage mutation rate in one or more of the bacteriophage-containing samples in response to the at least one first detectable parameter. In some embodiments, one or more of the samples in the plurality of receptacles includes a population of bacteriophage, and the feedback controller is configured to adjust a selection goal of one or more of the bacteriophage-containing samples in response to one or more of the at least one first detectable parameter. In some embodiments, the sensor is configured to detect at least a first detectable parameter and a second detectable parameter in one or more of the samples in the plurality of receptacles; and wherein the feedback controller is configured to make one or more real-time adjustments in the one or more of the samples based at least in part on the first detectable parameter and the second detectable parameter. In some embodiments, the means of detecting the first and second detectable parameters includes detecting a first and a second detectable agent, respectively. In certain embodiments, the first detectable agent includes a first fluorescent molecule and the second detectable agent includes a second fluorescent molecule. In some embodiments, one or more of the samples in the plurality of receptacles includes a population of bacteriophage. In some embodiments, one or more of the samples in the plurality of receptacles includes a polymerase. In some embodiments, the polymerase includes a T7 RNA polymerase. In certain embodiments, the first detectable agent includes an mCherry molecule and its presence in the sample indicates the first characteristic of positive selection in the sample and the second detectable agent includes a GFP molecule and its presence in the indicates the second characteristic of negative selection in the sample; and wherein the feedback controller is configured to adjust one or more of the samples in the plurality of receptacles in real time, based at least in part on the determination of at least one of the first characteristic and the second characteristic. In some embodiments, the first detectable agent includes a first luminescent molecule and the second detectable agent includes a second luminescent molecule. In some embodiments, the feedback controller is configured to adjust one or more of the samples in real time by adding an inducing agent to one or more of the samples in the plurality of receptacles. In certain embodiments, the inducing agent includes at least one of L-arabinose, anhydrotetracycline, theophylline, 2,4-diacetylphophloroglucinol, cuminic acid, 3-oxohexanoyl-homoserine lactone, vanillic acid, isopropyl β-D- 1-thiogalactopyranoside (IPTG), choline chloride, naringenin, 3,4-dihydroxybenzoic acid, sodium salicylate, and 3-hydroxytetradecanoyl-homoserine lactone. In some embodiments, the system also includes at least one temperature element configured to selectively modulate a temperature of one or more of the samples in the plurality of receptacles. In some embodiments, the feedback controller is configured to activate the temperature element in real-time based at least in part on a temperature of one or more of the samples in the plurality of receptacles. In some embodiments, the feedback controller is configured to activate the temperature element to modulate the temperature in real-time to hold at 37° C. In certain embodiments, the feedback controller is configured to activate the temperature element to modulate the temperature in real time to a temperature between one or more of: 25° C. and 40° C., 30° C. and 40° C., 32° C. and 38° C., 34° C. and 37° C., and 34.5° C. and 37.5° C. In some embodiments, the liquid handling robot further includes one or more reservoirs, wherein each reservoir is configured to hold an independently selected liquid exchange fluid. In some embodiments, the independently selected liquid exchange fluid includes a bacteriophage solution. In some embodiments, the liquid handling robot is configured to selectively dispense the bacteriophage solution into one or more receptacles in the plurality of receptacles. In certain embodiments, the independently selected liquid exchange fluid includes an inducing agent. In some embodiments, the reservoir has at least four ports including a first port to fluidly couple the reservoir to a water line, a second port to fluidly couple the reservoir to a cleaning fluid line, a third port to fluidly couple the reservoir to a liquid exchange fluid line, and a fourth port to fluidly couple the reservoir to a drain line. In some embodiments, the liquid handling robot further includes one or more peristaltic pumps configured to selectively perform liquid exchange in one or more of the samples in the plurality of receptacles. In certain embodiments, each sample in the plurality of receptacles is contained in a separate receptacle. In some embodiments, the system includes one or more sets of receptacles, wherein a set includes at least 1, 5, 10, 15, 20, 30, 40, 50, 60, 70, 80, 90, 100, 150, 200, 250, 300, 350, 400, 450, 500, 550, 600, 650, 700, 750, 800, 850, 900, 950, 1000 or more receptacles. In some embodiments, the system includes at least 1, 2, 3, 4, 5, 6, 7, 8, 9, 10, 15, 20, 25, or more sets of receptacles, each with an independently selected number of receptacles. In certain embodiments, one or more of the sets of receptacles includes 96, 192, or 384 receptacles. In some embodiments, the sensor is configured to detect at least one parameter of all of the samples within one or more of the sets of receptacles. In some embodiments, the feedback controller is configured to make at least one real-time adjustment in all of the samples within one or more of the sets of receptacles based upon the at least one detectable parameter. In some embodiments, the detecting includes transferring an aliquot of each of the one or more samples in the plurality of receptacles into a separate detection receptacle, and wherein the sensor is configured to detect at least one parameter of the transferred aliquot while the transferred aliquot is in the detection receptacle. In certain embodiments, the one or more samples includes at least two samples. In some embodiments, the one or more samples includes a plurality of samples. In some embodiments, the one or more cells includes at least two cells. In some embodiments, the one more cells includes a plurality of cells. In certain embodiments, the one or more horizontally transferable nucleic acids includes 2, 3, 4, 5, 6 or more independently selected horizontally transferable nucleic acids.

According to another aspect of the invention, a method for evaluating the suitability of diverse engineered cells to accomplish directed evolution is provided, the method including: providing one or more receptacles in a plurality of receptacles, with one or more samples, wherein (i) each sample is in a separate receptacle in the plurality of receptacles; (ii) one or more independently selected receptacles in the plurality of receptacles includes a cell; and (iii) one or more independently selected receptacles in the plurality of receptacles includes one or more horizontally transferable nucleic acids; providing a liquid handling robot configured to perform liquid exchange in one or more receptacles in the plurality of receptacles; facilitating mixing in one or more samples in the plurality of receptacles; detecting at least a first detectable parameter in one or more of the samples in the plurality of receptacles; and modifying one or more samples in the plurality of receptacles in real-time based at least in part on the first detectable parameter of the one or more samples; wherein: (i) at least one of the horizontally transferable nucleic acids is preselected to be evolved; (ii) one or more of the samples in the plurality of the receptacles includes one or more of the horizontally transferable nucleic acids preselected to be evolved; (iii) one or more of the samples in the plurality of receptacles does not include the one or more horizontally transferable nucleic acids preselected to be evolved; (iv) at least one cell in one or more of the samples in the plurality of receptacles is an engineered cell encoding one or more independently selected engineered alterations capable of determining an activity of at least one of the horizontally transferable nucleic acids preselected to be evolved; and wherein (v) detecting the at least one first detectable parameter includes detecting the activity determined by the one or more independently selected engineered alterations. In some embodiments, the mixing is within the receptacle. In some embodiments, the facilitated mixing includes mixing (a) one or more of the samples not comprising a horizontally transferable nucleic acid and (b) one or more of the samples comprising a horizontally transferable nucleic acid. In certain embodiments, the horizontally transferable nucleic acids are bacteriophages, including filamentous phages such as M13/f1/fd, temperate phages such as lambda, or lytic phages such as T1-T7. In some embodiments, the horizontally transferable nucleic acids are conjugative plasmids, such as F, F0lac, Col1b, or RP4/RK2. In some embodiments, the horizontally transferable nucleic acids are secreted by at least a portion of the cells in one or more of the samples, wherein the means of secreting includes a DNA export system. In some embodiments, the DNA export system includes a N. gonorrhoeae DNA export system. In certain embodiments, the secreted nucleic acids are internalized by at least a portion of the cells in one or more of the samples, wherein the internalization means includes a DNA transporter or other bacterial competence machinery. In some embodiments, the horizontally transferable nucleic acids encode one or more genes that produce one or more biomolecules that exhibit at least one direct sensor-detectable parameter. In some embodiments, one or more of the cells produce an indirect sensor-detectable parameter when triggered by the production of one or more biomolecules encoded by the horizontally transferable nucleic acids. In certain embodiments, a feedback controller is also provided and is configured to make at least one real-time adjustment in one or more of the samples in the plurality of receptacles based upon the at least one detectable parameter. In some embodiments, a sensor is also provided and is configured to detect at least one detectable parameter of one or more of the samples while the one or more samples are in the receptacles. In some embodiments, the detection of the detectable parameter determines a characteristic of one or more of the samples in the plurality of receptacles. In some embodiments, one or more of the samples in the plurality of receptacles includes a population of bacteriophage. In certain embodiments, the determined characteristic includes at least one of: selection, positive selection, negative selection, an enzyme activity, phage population size, bacterial population size, gene transcription, and biomolecule evolution in one or more of the samples in the plurality of receptacles. In some embodiments, the detecting includes detecting one of more of a presence, an absence, and an amount of the detectable parameter. In some embodiments, the method also includes determining a characteristic of one or more of the samples, based at least in part on the first detectable parameter. In some embodiments, the determined characteristic includes at least one of: selection, positive selection, negative selection, an enzyme activity, phage population size, gene transcription, biomolecule evolution, and promoter activity in the one or more samples. In certain embodiments, the detecting includes detecting one of more of a presence, an absence, and an amount of the first detectable parameter. In some embodiments, the first detectable parameter includes one of more of absorbance, luminescence, and fluorescence. In certain embodiments, one or more of the samples in the plurality of receptacles is modified in real-time based upon at least one of the detected absorbance, luminescence, and fluorescence. In some embodiments, one or more of the samples comprise a bacteriophage population, and wherein the modification of one or more samples in the plurality of receptacles is selected for one or more of: (a) modifying a positive selection strength of at least one sample comprising a bacteriophage population in response to the first detectable parameter; (b) modifying a negative selection strength of at least one sample comprising a bacteriophage population in response to the first detectable parameter; (c) modifying a mutation rate of at least one sample comprising a bacteriophage population in response to the first detectable parameter; and (d) modifying a selection goal of at least one sample comprising a bacteriophage population in response to the first detectable parameter. In some embodiments, modifying one or more of the samples in the plurality of receptacles includes adding an inducing agent to one or more of the samples in the plurality of receptacles in response to the first detectable parameter. In some embodiments, the method also includes detecting a second parameter in one or more of the samples in the plurality of receptacles; and modifying one or more of the samples in the plurality of receptacles in real-time based at least in part on the second detectable parameter of the one or more samples. In certain embodiments, the method also includes providing one or more reservoirs, wherein each reservoir is configured to hold and selectively dispense an independently selected liquid exchange fluid; sterilizing one or more of the reservoirs with a solution comprising bleach; and placing one or more of the independently selected liquid exchange fluids into a selected reservoir of the one or more reservoirs. In some embodiments, at least one of the independently selected liquid exchange fluids includes a bacterial culture. In some embodiments, at least one of the independently selected liquid exchange fluids includes a bacteriophage solution. In some embodiments, at least one of the independently selected liquid exchange fluids includes an inducing agent. In some embodiments, at least one of the one or more reservoirs has at least four ports including a first port to fluidly couple the reservoir to a water line, a second port to fluidly couple the reservoir to a cleaning fluid line, a third port to fluidly couple the reservoir to a liquid exchange fluid line, and a fourth port to fluidly couple the reservoir to a drain line. In certain embodiments, the liquid handling robot further includes one or more peristaltic pumps configured to selectively perform liquid exchange in one or more of the samples in the plurality of receptors. In some embodiments, each sample is in a separate receptacle and the method includes a plurality of samples each in a separate receptacle. In some embodiments, the sterilizing step includes: washing the reservoir with a bleach solution comprising at least 8%, 9%, 10%, 11%, or 12% bleach; and rinsing the reservoir with a solution comprising water. In certain embodiments, the bleach solution includes 10% bleach. In some embodiments, the rinsing step is performed at least 2, 3, 4, 5, 6, 7, 8, 9, 10 or more times. In some embodiments, the detecting step includes transferring an aliquot of one of the samples from the receptacle containing the sample into a detection receptacle, and detecting the at least one parameter of the aliquoted sample while the aliquot is in the detection receptacle. In some embodiments, the modifying step includes modifying one or more of the samples within the receptacles in real-time based upon the first detectable parameter. In certain embodiments, the method includes use of any embodiment of any aforementioned aspect of the invention. In some embodiments, the one or more samples includes at least two samples. In some embodiments, the one or more samples includes a plurality of samples. In certain embodiments, the one or more cells includes at least two cells. In some embodiments, the one more cells includes a plurality of cells.

According to yet another aspect of the invention, methods of conducting continuous directed evolution are provided, wherein the means for conducting the continuous directed evolution includes a system set forth as any embodiment of any aforementioned aspect of the invention.

According to yet another aspect of the invention, methods for determining the suitability of diverse engineered cells to accomplish directed evolution are provided, wherein the means for determining the suitability of the diverse engineered cells includes a system set forth in any embodiment of any aforementioned aspect of the invention.

### BRIEF DESCRIPTION OF THE DRAWINGS

FIG. 1 is a schematic diagram showing advantages of robotic PACE. The drawing shows the relationship between new capabilities of one embodiment of the robotic platform (top row) and their corresponding advantages. The drawing illustrates certain advantages in an embodiment of a system for conducting continuous evolution and a system for evaluating diverse engineered cells to accomplish directed evolution.

FIG. 2 is a schematic view of one embodiment of a system for conducting continuous evolution. The embodiments include a liquid handling robot to transfer liquids, rather than tubes and pumps. The schematic is also an embodiment of a system for evaluating diverse engineered cells to accomplish directed evolution.

FIG. 3A-C provides schematic views of another embodiment of one or both of a system of the invention, which is suitable for conducting continuous evolution and/or evaluating diverse engineered cells to accomplish directed evolution. FIG. 3A shows carboys included a waste carboy and a media-containing carboy. FIG. 3B shows a bacterial chemostat, which is in an incubator to maintain appropriate temperature. FIG. 3C shows receptacles (also referred to herein as lagoons) and a reservoir +arduino, which are illustrated in the context of a robotic element of the system.

FIG. 4 is a schematic view of a reservoir according to an embodiment of a system for conducting continuous evolution. The illustrated reservoir may also be used in certain embodiments of a system of the invention for evaluating diverse engineered cells to accomplish directed evolution.

FIG. 5 is a schematic view of a reservoir according to one embodiment of a system of the invention for conducting continuous evolution. The illustrated reservoir may also be used in some embodiments of a system of the invention for evaluating diverse engineered cells to accomplish directed evolution.

FIG. 6 is a schematic view of a portion of a liquid handling robot including a plurality of pumps that may be included in some embodiments of systems of the invention and used to conduct continuous evolution. The illustrated portion of a liquid handling robot may also be included in some embodiments of systems of the invention for evaluating diverse engineered cells to accomplish directed evolution.

FIG. 7A-J provides graphs of results from ten experimental studies in which wild type T7 RNAP was evolved to bind the T3 promoter in an embodiment of the invention utilizing a liquid handling robot with 10 simultaneous phage populations. FIG. 7A-G, FIG. 7I, and FIG. 7J are results from phage-containing experiments. FIG. 7H shows results of the no-phage control. Each graph shows the real-time luminescence readout [corrected for absorbance, in Absorbance Units (AU)], a proxy for the populations'activity on the T3 promoter.

FIG. 8A-C provides graphs of experimental results for tip re-use and sterilizing the reservoir in an embodiment of the invention. Luminescence monitoring for three containers, two no-phage controls (FIG. 8A and FIG. 8C) and one that contained phage (FIG. 8B). The results confirm that no cross-contamination occurred.

FIG. 9A-B provides graphs of experimental results using real-time monitoring in a system of the invention. The real-time monitoring of robotic PACE is show in FIG. 9A with real-time readout of luminescence/absorbance and corresponding plaque assays (FIG. 9B). FIG. 9B shows the total phage titer (squares) and activity dependent phage title in (circles).

FIG. 10A-B provides a schematic representation of the feedback controller of a system for conducting continuous evolution according to one embodiment. FIG. 10A illustrates an experiment utilizing two readouts: mCherry and positive selection and GFP for negative selection. Three small molecules control it: l-arabinose controls mutagenesis; anhydrotetracycline modulates positive selection; and a theophylline-sensitive riboswitch controls negative selection. FIG. 10B illustrates incorporation of real-time monitoring of the two readouts into robotic control over all three chemical induces, as well as providing different bacterial cultures.

FIG. 11A-B provides a schematic diagram showing constructs for intron selection. FIG. 11A shows that pIII is split by an intron, and only the C terminal side is encoded in the phage genome and can evolve. The exon sequence is marked with vertical arrow at the end of the top arrow head. FIG. 11B shows the splicing reactions that occur to produce full-length pIII protein.

FIG. 12A-B provides a schematic diagram showing an embodiment of an embedded intron strategy. FIG. 12A shows a second intron is used so that both exon sequences, marked with vertical open arrows, are encoded in the static bacterial genome. FIG. 12B shows the splicing reactions that occur to produce full-length pIII protein. The intron (shown on the right) splices first forming the full-length version of the Intron-C′pIII, which is then challenged to splice.

FIG. 13A-B provides a schematic diagram showing an embodiment of constructs for intein selection. FIG. 13A shows constructs for evolving tolerance onto the extein sequence, marked with vertical arrows, on the N-terminal side. This construct includes one intron. The intein is involved such that it tolerates the new extein sequence. FIG. 13B shows that upon phage entry in the cell, the intein is challenged to splice a full-length pIII.

FIG. 14 provides a schematic diagram showing an embodiment of constructs for simultaneous evolution of extein compatibility on both the C and N terminal sides. The vertical arrows indicate the exon sequences.

FIG. 15 provides an image of an embodiment of a system of the invention. Not shown is the right-hand side of deck enclosure, which covers the plate reader.

FIG. 16 provides an image of an embodiment of a system of the invention and includes an off-deck chemostat. Peristaltic pumps and stir plate are housed inside of a 37^{o}C incubator. At right are the LB and waste carboys.

FIG. 17A-B provides schematic diagrams of different embodiments of monitoring and selection procedures. FIG. 17A illustrates embodiments in which a desired biomolecule activity is indirectly tied to pIII production and which may include monitoring other cellular signals. FIG. 17A illustrates evolving an enzyme “*”, which participates in the biosynthetic pathway (shown on the right), in order to increase production of X. One or more readouts of GFP, luxAB, and pIII can be performed at different locations in the procedure. FIG. 17B illustrates a means for generalized phage titer monitoring that is unrelated to selection performance.

FIG. 18A-D provides schematic diagrams of various monitoring and selection procedures. FIG. 18A shows a selection embodiment in which the desired biomolecule activity results in transcription of pIII. FIG. 18B-D shows selection embodiments in which the desired biomolecule activity results in translation of pIII. FIG. 18B illustrates an embodiment comprising monitoring activity in Trans by enabling readout. FIG. 18C illustrates an embodiment comprising monitoring activity in Trans by disabling readout, and FIG. 18D illustrates an embodiment comprising monitoring activity in Cis.

FIG. 19 provides a diagram of a fluorescence plot showing fluorescence versus the length of time since inoculation with phage. The traces (from left to right traces #1- #6), illustrate the following titers: Trace 1 titer is 10⁵, Trace 2 titer is 10⁴, Trace 3 titer is 10³, Trace 4 titer is 10², Trace 5 titer is 10¹, and Trace 6 titer is 10⁰.

FIG. 20A-B provides graphs of results from three parallel robotic directed evolution experiments in which T7 RNAP was evolved to bind the T3 promoter in an embodiment of the invention utilizing a liquid handling robot. FIG. 20A shows plaque assay analyses of samples taken from the three experiments over time. As the population became able to propagate on the evolutional goal, the total phage titer dropped and then recovered. In the no-phage control experiment (middle panel), no phage are observed for the duration of the experiment, confirming that cross-contamination had not occurred. FIG. 20B shows real-time luminescence monitoring of the experiments in FIG. 20A. The results confirm that luminescence monitoring data track the activity-dependent plaque assay results.

FIG. 21A-B provides a schematic representation of the experimental design of and graphs of results for 48 parallel robotic directed evolution experiments in which tRNAs were evolved to decode a TAGA quadruplet codon. The 48 parallel robotic directed evolution experiments were performed using an embodiment of the invention utilizing a liquid handling robot. FIG. 21A shows a schematic of a phage propagation reporter, a schematic of a selection phage construct, and a schematic of the directed evolution selection strategy. FIG. 21B shows graphs of results obtained from absorbance and luminescence monitoring during an embodiment of robotic continuous directed evolution. The left panel shows real-time absorbance and the right panel shows luminescence monitoring of parallel robotic directed evolution experiments in nine lagoons of interest. Lagoons with high phage titer showed depressed luminescence, which was used to identify high-activity lagoons as well as lagoons that had evolved recombinants. As shown in FIG. 21B, right panel, a previously evolved phage, p241, immediately triggered activity-dependent luminescence monitoring. Other lagoons required evolution in order to identify an evolution solution, and results shown in FIG. 21B showed a rise in luminescence detected in lagoons over time.

### DETAILED DESCRIPTION

The invention, in part, includes systems and methods that significantly improve continuous evolution procedures and the quality and scope of results that can be obtained with such approaches. One known continuous evolution methodology that can be enhanced using methods and systems of the invention is Phage Assisted Continuous Evolution (PACE). PACE is a directed evolution technology that leverages the fast life cycle of bacteriophages to perform rapid protein evolution. As a non-limiting example, traditional PACE procedures may include methods in which an essential gene encoding the tail fiber pIII is removed from the M13 bacteriophage genome and replaced with gene(s) encoding a biomolecule of interest to be evolved. The phages are then challenged to replicate in bacteria engineered to detect the desired molecular activity and produce pIII in response. This “selection circuit” can trigger an arms race in the phage population to evolve the researcher's biomolecule(s) of choice. Embodiments of systems and methods of the invention may be used for continuous directed evolution, which is a general method capable of evolving specific biomolecule, by evolving at the level of the encoding gene, over many cycles of mutation, selection, and replication. Systems and methods of the invention can be used for different selections, including those set forth herein and also others known and practiced in the art using traditional PACE methods including, but not limited to those set forth herein.

Known benefits of traditional PACE methods include the ability of included phage to replicate autonomously every 20-60 minutes, permitting performance of many more rounds of evolution per day than were possible with previous discrete evolution methods. In addition, evolving phage populations may have titers of approximately 10⁸ infected cells/mL, which permit more variants to be assessed than could be assessed using previous directed evolution approaches. Another advantageous aspect of traditional PACE procedures is that their use permits avoidance of most difficulties with ‘cheater’ mutations, because with traditional PACE, experimental conditions can be set such that the phage genome can evolve, but the bacteria are constantly washed out before they can replicate, allowing everything encoded by the bacterial genome to be considered ‘fixed.’

Application of systems and methods of the invention to traditional PACE can increase the ability of PACE to optimize binding interactions between DNA, RNA, and/or protein via an n-hybrid approach, a technique in which binding interactions between “N” biomolecules are detected, in some instances using a proximity-dependent readout. Compared to traditional PAC, embodiments of systems of the invention can be used to obtain enhanced results when evolving protein and RNA therapeutics and molecular probes, as well as when evolving many other molecular tools relevant to health applications and research. Widespread applications for traditional PACE are known and practiced in the art, including but not limited to procedures to evolve proteases, evolve DNA-binding proteins, and to evolve protein-protein interactions. In addition to protein engineering applications, traditional PACE systems and methods can also be used as tools for the study of evolution, a non-limiting example of which is the use of traditional PACE methods to predict how pathogens will escape from protease drug-inhibitor therapeutics.

Systems and methods of the invention can be used to implement continuous evolution in a discretized manner to perform PACE using discrete components of the system. The discretized nature of embodiments of systems and methods of the invention renders them capable of overcoming various limitations of traditional PACE methods. Traditional PACE methods face a number of difficulties, including limitations due to the extensive tuning of construct and inducer concentrations that are required to develop an evolution circuit that selects strongly in favor of a desired activity. In addition, traditional PACE is limited by a lack of reproducibility of experimental outcomes, including success or failure, which depend on the choice of experimental conditions such as the mutation rate and which intermediate selection goals are used. Traditional PACE systems are also limited in the ability to scale because they cannot carry out more than a handful of experiments at once because they rely on peristaltic pumps and tubing to culture the phage populations, an arrangement that does not scale. Traditional PACE methods are not amenable to running more than very few simultaneous PACE experiments at one time, at least in part because of the inability to compare the different populations. Furthermore, traditional PACE does not include monitoring activity of each evolving population in real-time, resulting in a need for practitioners to try conditions nearly blindly and observe outcomes only after the fact.

Aspects of the present disclosure are directed to solving one or more of these above technical limitations of traditional PACE. It has now been recognized that these longstanding technical problems may be solved by performing many parallel instances of the same evolution experiment in order to explore a variety of experimental conditions and inducer concentrations and to utilize real-time monitoring and feedback control over experimental conditions. As used herein, the term “inducer” is used interchangeably with the term “inducing agent”. Embodiments of systems and methods of the invention also take in to account that optimized conditions for successful molecular evolution are not constant, but depend on the current molecular activity in samples. For example, a population at low activity will be lost, or washed out if subjected to a high-stringency selection, and a population trapped a local fitness optimum would benefit from temporarily relaxing selection strength or a changing the selection goal. Embodiments of systems and methods of the invention permit flexibility in determining and producing, and optimizing conditions for continuous evolution, including PACE procedures. Aspects of the invention may be used to enhance prior PACE methods, at least in part, based on embodiments of systems and methods of the invention that permit a practitioner to monitor the status of each independently evolving population in real-time and make real-time adjustments to adjust and/or optimize the continuous evolution conditions and outcome.

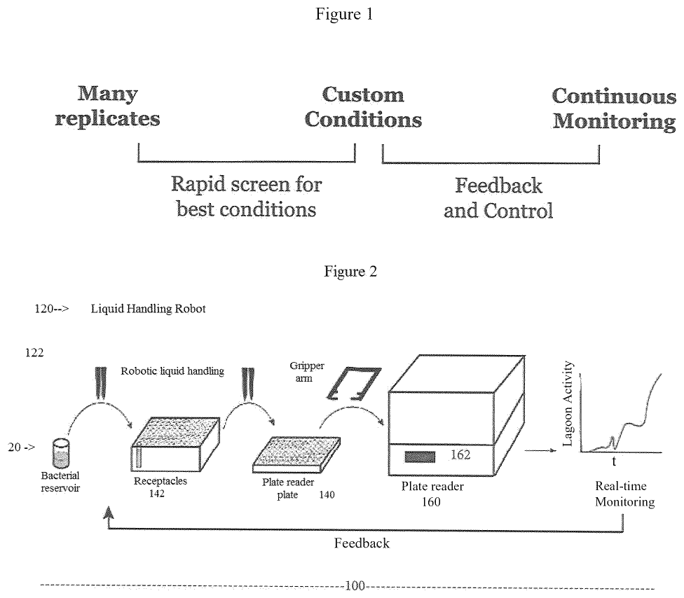

As illustrated in FIG. 1 and as set forth in further detail elsewhere herein, one aspect of the present disclosure is directed to systems and methods for conducting discretized continuous directed evolution that are configured to make many replicates, provide custom conditions, and/or provide continuous monitoring capabilities. In addition, certain embodiments of systems of the invention may additionally be configured for real-time adjustments to conditions in one or more of the replicates. As shown in FIG. 1, a system that has the ability to make many replicates with custom conditions enables rapid screening to identify different conditions, which in certain embodiments of the invention include the most optimal conditions. Furthermore, a system that has the ability to provide custom conditions and continuous monitoring permits feedback and control, including but not limited to real-time control, over the system. Parameters of systems and methods of the invention may be monitored and can be adjusted in real time to optimize conditions beneficial for the desired evolution. The processes of evolving one or more genes of interest can be run in a plurality of receptacles in parallel with the use of a liquid handling robot that is capable of independently altering the content and condition for one or more receptacles in the system. In some embodiments of the invention one or more conditions in one or more receptacles are detected and assessed (using one or more detectors) and the information is applied in a manner that conditions such as temperature, receptacle contents, etc. may be adjusted in one or more receptacles. It will be understood that adjustments can be made in a single receptacle or in a plurality of receptacles and each adjustment can be independently selected so that a variety of different conditions can be used in parallel continuous evolution preparations using systems and methods of the invention.

As set forth in more detail below, in an aspect of the present disclosure's systems and methods for conducting discretized continuous directed evolution are provided. Also, in another aspect of the invention, systems and methods for evaluating suitability of engineered cells to accomplish directed evolution are provided. Certain embodiments of each of these systems and methods may be configured to run in a multi-well plate format using a liquid handling robot. In one embodiment, the system is configured to run up to hundreds or thousands of separate populations, (non-limiting examples of which may be phage populations and cell populations), in a simultaneous manner; customizing the conditions in each experiment; and monitoring the fitness of each population. As non-limiting examples, the new technical capabilities of embodiments of systems and methods of the invention enable researchers to (i) screen for experimental conditions that are suited to the particular evolution they are attempting; (ii) run from 2, 3, 4, 5, 6, 7, 8, 9, 10, 50, 100, 500, up through massively multiplexed samples and experiments; and (iii) to monitor and respond to the progress of each sample and experiment as it evolves in real time.

Systems and methods of the invention may be used to obtain data from simultaneous populations undergoing continuous evolution, which is of considerable value in research. For example, if a mutation reaches fixation in three or more populations, it is more likely to be functionally important than a mutation that fixes in just one population, which make it desirable to be able to collect data from multiple populations. Embodiments of systems and methods of the invention may be used to run many replicate experiments simultaneously and obtain and utilize data from the replicates. Non-limiting examples of applications for which embodiments of systems and methods of the invention can be used are for identification and assessment of: epistatic interactions, hitchhiker mutations, and/or functional mutations that are often important to adapt an evolved biomolecule to a target organism.

Additionally, certain embodiments of systems, components, and methods of the invention may be used to run PACE procedures that begin with a plurality of different initial biomolecules without forcing them through a competitive bottleneck that could constrain future outcomes. Systems and methods of the invention can be successfully used in directed evolution experiments started from libraries of recombinant, or de novo biomolecules. Unlike previous methods in which only a very limited number of PACE experiments could be effectively run at one time, embodiments of systems and methods of the invention can use, and benefit from use of a computationally designed starting library.

As set forth herein, various aspects of the present disclosure are directed to combining continuous evolution with a robotic liquid-handling platform that may be configured to monitor a plurality of evolving populations in real time. In one embodiment, the system may include on-deck sterilizing and re-use of components to enable from minimal through massively parallel directed evolution. In certain embodiments, the system may leverage real-time monitoring data to dynamically optimize conditions in directed evolution experiments. Furthermore, in some embodiments, the system may create a high-throughput pipeline for evolving efficient RNA-editing introns. These and other concepts are discussed in greater detail elsewhere herein.

General concepts and practice of traditional continuous evolution are well known and routinely used in the art. Certain embodiments of continuous directed evolution systems and methods of the invention disclosed herein incorporate methods and components of traditional PACE methods, for example those disclosed in U.S. Pat. Nos. 9,394,537 and 9,771,574, the content of each of which is incorporated by reference herein in its entirety. The following provides information on various embodiments of the invention including an overview of applications, system components, and other elements used in various embodiments of systems and methods of the invention.

### Applications

Systems and methods of the invention can be used for numerous applications to assess elements such as, but not limited to, molecules, pathways, activities, interactions, and properties, some of which have previously be assessed using traditional PACE methods. Systems and methods of the invention can be used to more effectively assess these and other types of pathways, molecules, systems, and characteristics, including but not limited to:
elements such as binding, recognition, and interactions between molecules, etc. It will be understood that embodiments of the invention provide high-throughput directed continuous evolution systems and methods that enhance the efficiency, effectiveness, and use of previous directed evolution methods.

Non-limiting examples of applications in which embodiments of systems and methods of the invention can be used, are: protease substrate recognition [see for example: Packer, Michael S. et al. 2017. Nature Communications 8 (1):956.]; protease substrate characterization [see for example: Dickinson, Bryan C. et al. 2014. Nature Communications 5 (October):5352]; protein-protein binding [see for example: Badran, Ahmed H. et al. 2016. Nature 533 (7601):58-63 and Pu, Jinyue et al. 2017. Nature Chemical Biology 13 (4): 432-38]; polymerase promoter recognition [see for example: Esvelt, Kevin M. et al. 2011. Nature 472 (7344):499-503, Dickinson, Bryan C. et al. 2013. PNAS USA 110 (22):9007-12, Leconte, Aaron M. et al. 2013. Biochemistry 52 (8):1490-99, and Badran, Ahmed H. & Liu, D. R., 2015. Nature Communications 6 (October):8425]; Aminoacyl tRNA synthetases, for charging different tRNA scaffolds, or charging with non-natural amino acids [see for example Bryson, David I. et al. 2017. Nature Chemical Biology, October. //doi. org/10.1038/nchembio.2474]; rRNA; Properties of CRISPR systems; Enzymatic pathways; introns and inteins; and properties of optogenetic proteins. All of the above-cited references are all hereby incorporated by reference in their entirety. It will be understood that methods and systems of the invention can be applied to applications listed above and one skilled in the art, will understand how to apply embodiments of systems and methods disclosed herein for these and other applications. Traditional PACE procedures, components, and methods are known and routinely used in the art, see for example, references cited above and U.S. Pat. Nos. 9,394,537, 9,771,574; and Dickson, B. C. et al. Nat. Comm. Oct. 20, 2014, DOI: 10.1038/ncomms6352, the content of each of which is incorporated by reference herein in its entirety. Aspects of the present disclosure are directed to systems, components, and methods for conducting directed evolution that may be applied to problems previously intractable using traditional PACE technology.

### General Overview of System Components

Systems of the invention comprise various components, non-limiting examples of which include one or more: receptacles, liquid-handling robots, sensors, integrated detectors, feedback controllers, non-integrated detectors, temperature controller elements, reservoirs, bacteriophage solutions, inducing agents, reservoir ports, fluid lines, peristaltic pumps, sets of receptacles, detection receptacles, samples, cells, host cells, and horizontally transferrable nucleic acids. Information on various components is provided herein and it will be understood that art-known components of traditional PACE and other continuous evolution methods and procedures may also be utilized in embodiments of systems and methods of the invention.

### Receptacles

As used herein the term “receptacle” means a tube, vial, well (a non-limiting example of which is a well in a multiwall plate, a microplate, or a microtiter plate) or other suitable holder configured to contain a sample comprising cells and for liquid exchange. The term “receptacle” is used interchangeably herein with the term “lagoon.” A configuration of a receptacle used in a method or system of the invention is suitable for actions such as, but not limited to: mixing of the receptacle's contents, transferring fluid into and out of the receptacle, detecting parameters of samples in the receptacle, etc. A receptacle used in a method and/or system of the invention may be (i) configured to contain, and/or (ii) contains one or more horizontally transferable nucleic acids.

A receptacle used in a system and/or method of the invention may be made of any suitable material, non-limiting examples are polystyrene, polypropylene, polycarbonate, cyclo-olefins, glass, quartz or other suitable art-known material. A receptacle used in an embodiment of a system or method of the invention may be clear, white, or another color, as suitable for detection methods. In some embodiments of the invention a receptacle is a color suitable for optical absorbance and/or luminescence detection. Receptacles used in an embodiment of a system and/or method of the invention may be opaque, black, or another dark color as appropriate for certain fluorescent detection methods. An embodiment of a system and/or method of the invention may include a plurality of receptacles. As used herein, in certain embodiments of systems and methods of the invention, a plurality of receptacles is one or more of: at least 3 receptacles; at least 5 receptacles; at least 10 receptacles; at least 15 receptacles; at least 50 receptacles; at least 100 receptacles; at least 500 receptacles; at least 1000 receptacles; and at least 10,000 receptacles. An embodiment of a system or method of the invention may include one or more sets

of receptacles. As used herein the term “set,” used in reference to receptacles is a group of more than one receptacle that are physically associated with each other. For example, a set may be a manufactured multiwell plate, a microplate, a microtiter plate—each of which comprises a plurality of receptacles. Some aspects of the invention include 1, 2, 3, 4, 5, 6, 7, 8, 9, 10, 20, or more sets of receptacles. As a non-limiting example, a set of receptacles used in an embodiment of a system or method of the invention is a 96-well plate.

The term “sample,” as used herein in reference to a continuous evolution system or method of the invention means components such as, but not limited to: a cell, a nucleic acid, a fluid, etc. that is included in one or more of an evolution procedure, a control procedure, analysis, testing, etc. As a non-limiting example, a sample in a system or method of the invention may comprise a fluid, one or more cells, one or more horizontally transferable nucleic acids, one or more inducing agents, or other suitable components. In certain embodiments of systems and methods of the invention, a sample is located in a receptacle and a liquid exchange robot is used to perform one or more of: adding liquid to the sample, preparing the sample, mixing the sample, removing the sample, removing one or more aliquots of a sample, transferring the sample, etc.

### Liquid Exchange and Handling

Another component included in embodiments of a system and/or used in a method of the invention is a liquid handling robot. Although the physical size, shape, etc. may differ in different embodiments of the invention, a liquid handling robot is configured to perform liquid exchange in one or more receptacles in the plurality of receptacles. A liquid handling robot used in a system or method of the invention may be configured to add material to a receptacle, remove material from a receptacle, mix a contents of a receptacle, remove an aliquot of a sample from a receptacle, as well as for other tasks. As used herein, the term “material” used in reference to a receptacle is used to refer to a sample, a portion of a sample, an aliquot, liquid, a solution, a suspension, cells, or other suitable inclusion in a receptacle or set of receptacles of the invention. Non-limiting examples of a liquid handling robot are provided elsewhere herein including in the drawings, drawing description, examples, and specification. It will be understood that alternative liquid handling robots suitable for use in systems and methods of the invention are commercially available, or can be individually prepared for use in a system or method of the invention.

A liquid handling robot suitable for used in embodiments of systems and methods of

the invention comprises one or more reservoirs, wherein each reservoir is configured to hold an independently selected fluid. A fluid in a reservoir of the invention may be a “liquid exchange fluid,” in that it is configured for one or more of: being added to and being mixed with one or more samples in a plurality of receptacles. A fluid in a reservoir may comprise bacteriophage, and be referred to herein as a “bacteriophage solution”. A liquid-handling robot may be configured to selectively dispense a liquid, a non-limiting example of which is a bacteriophage solution, into one or more receptacle in a system of the invention. It will be understood that one or more of the receptacles into which a bacteriophage solution is added using a liquid handling robot of the invention may also include other materials. For example, in some embodiments of the invention a receptacle comprises one or more of an additional: liquid, solution, suspension, chemical, nucleic acid, polypeptide, and compound before or after addition of a bacteriophage solution by the liquid-handling robot of the invention.

Another non-limiting example of an independently selected liquid-exchange fluid that can be included in one or more liquid-handling robot reservoirs is a fluid comprising one or more inducing agents. It will be understood that an inducing agent or inducer, is an agent and/or small molecule that increases the rate of a gene mutation. Thus, in some embodiments of the invention, a host cell population may be contacted with an inducer for an inducible promoter in an amount sufficient to result in an increased rate of mutagenesis. As a non-limiting example, a bacterial host cell population is provided in which the host cells comprise a mutagenesis plasmid that includes an expression cassette and the expression cassette is controlled by an arabinose-inducible promoter. Thus, when the population of host cells is contacted with the inducer, for example, arabinose, it induces an increased rate of mutation.

Inducing agents can be delivered into one or more independently selected receptacles in a system or method of the invention, and non-limiting examples of inducing agent that may be used in embodiments of systems and methods of the invention are: L-arabinose, anhydrotetracycline, theophylline, 2,4-diacetylphophloroglucinol, cuminic acid, 3-oxohexanoyl-homoserine lactone, vanillic acid, isopropyl β-D-1-thiogalactopyranoside (IPTG), choline chloride, naringenin, 3,4-dihydroxybenzoic acid, sodium salicylate, and 3-hydroxytetradecanoyl-homoserine lactone.

Activity of a robot of the invention, such as a liquid handling robot may be referred to herein with the term “operation.” As used herein in reference to activity of a robot means an instance in which the robot manipulates the contents of one or more receptacles. The term operation may be used to refer to manipulating the contents of a single receptacle or manipulating the contents of a plurality of receptacles in the same or in different ways. One non-limiting example of an operation is a robot dispensing an inducer into a receptacle. Another non-limiting example is a robot dispensing an inducer into a plurality of receptacles. It will be understood that each robot operation may be independently selected with respect to the manipulation parameters. The term “manipulation parameter” as used herein, includes but is not limited to: an amount of an addition or removal from the contents of a receptacle, the timing of a manipulation of a receptacle, a frequency of a manipulation of a receptacle, the content of an addition into a receptacle, etc. It will be understood that each manipulation parameter in an embodiment of a system or method of the invention may be independently selected. In non-limiting examples, in some embodiments each manipulation parameter may be the same for two or more receptacles and in certain embodiments one or more manipulation parameters may be different for two or more receptacles. It will be understood that in some aspects of systems of the invention, one or more operations carried out by a system robot may be controlled with respect to timing, frequency and other manipulation parameters using software, a non-limiting example of which is scheduling software. It will be understood by those in the art how to select, design, and/or implement scheduling software in an embodiment of a system or method of the invention.

A reservoir in a system or method of the invention may include one or more ports, each of which fluidly couples the reservoir to a liquid agent or connects the reservoir to an element such as a line, which is configured to permit draining of liquid from the reservoir. As used herein the term “liquid agent” used in reference to a reservoir means a liquid that can be introduced into the reservoir. As a non-limiting example, an embodiment of a system of the invention includes a reservoir with at least four ports including a first port that fluidly couples the reservoir to a water line; a second port that fluidly couples the reservoir to a cleaning fluid line; a third port that fluidly couples the reservoir to a liquid exchange fluid line, and a fourth port that fluidly couples the reservoir to a drain line.

An advantage of using a liquid handling robot in systems and methods of the invention is flexibility in independently preparing and adjusting the content of each receptacle in the system or method. Thus, each receptacle and sample in a system of the invention can be independently monitored and adjusted, for example by detecting a parameter of a sample and adjusting the content of the sample with the liquid-exchange robot, which can deliver, remove, and/or mix the content of the sample in an independently selected manner. It will be understood that the adjustment actions by the liquid-exchange robot need not static or redundant, and can be independently changed, or left the same, across a selected given period of time. In some aspects of the invention an independently selected liquid exchange fluid comprises an inducing agent, examples of which are provided elsewhere herein. In certain aspects of the invention an independently selected liquid exchange fluid comprises one or more cells, host cells, bacterial cells, vectors, nucleic acids, polypeptides, or other materials desired to be delivered to, and/or mixed with a sample.

A function of a liquid handling robot of the invention is delivery, mixing, removal of various independently selected solutions, agents, etc. from a reservoir to one or more independently selected receptacles in a plurality of receptacles or one or more sets of receptacles. Functions such as liquid delivery, mixing, and removal from a receptacle may be performed using one or more pumps, a non-limiting example of which is a peristaltic pump. One or more peristaltic pumps may be included in a system and/or method of the invention and may be configured to selectively perform liquid exchange in one or more of the samples in the plurality of receptacles.

### Sensors and Detection

Another component that may be included in certain embodiments of systems and methods of the invention is a sensor configured to detect at least one detectable parameter of one or more of samples that are components in a system and/or method of the invention. In some aspects of the invention, one or more independently selected sensors are included. Non-limiting examples of a sensor that may be included in a system or method of the invention is a sensor configured to detect one or more of: absorbance, luminescence, fluorescence, and color in one or more samples. A sensor included in an embodiment of the invention may be constructed and arranged as an integrated plate reader. Alternatively, a sensor included in an embodiment of the invention need not be integrated but may be “remote.” It will be understood that a detection means used in a system and/or method of the invention may detect in a receptacle that is part of the system, or may detect in a different receptacle. For example, in some aspects of the invention at least a portion of a sample, also referred to herein as an aliquot, is removed from its receptacle and transferred into another

receptacle for detection of its contents with a sensor. The term, “detection receptacle” is used herein in reference to a receptacle into which a portion of a sample is placed and conveyed to a sensor for application of detection steps to that portion of the sample. It will be understood that results obtained from a portion of a sample can be extrapolated and thereby provide information about the sample not removed to the detection receptacle.

Non-limiting examples of detectors are described elsewhere herein including in the drawings, drawing descriptions, examples, written description, etc. It will be understood that alternative sensors are suitable for use in systems and methods of the invention, and are commercially available, or can be individually prepared for use in a system or method of the invention. In some aspects of the invention a sensor is a plate reader, which may be a plate reader that is integrated as part of the system of the invention, or may be a plate reader that is a component separate from the system, to which sample aliquots may be delivered for detection.

Timing and duration of detection of one or more samples can be independently adjusted and determined for one or more samples in some embodiments of systems and methods of the invention. As a non-limiting example, a sensor used in a system of the invention may be configured to detect over a predetermined time period of at least 1, 3, 6, 12, 18, 24, 36, 48, or more hours. Another detection element that can be adjusted for use in a system and/or method of the invention are intervals of detection. For example, a detector may be configured to detect one or more samples using one or more of the following parameters: (i) continuous detection over a period of time, (ii) interval detection over a period of time, and (iii) a combination of (i) and (ii) as desired by the operator of the system or method of the invention. Non-limiting examples of interval detection are (i) a sensor configured to detect once every 1, 5, 10, 15, 20, 30, 60, 120, or more minutes and use thereof, and (ii) a sensor configured to detect at a regular interval over 1, 3, 6, 12, 18, 24, 36, 48, or more hours and use thereof. Additional detection parameters will be known in the art and may be suitable for inclusion and use in a system and/or method of the invention. Those in the art will understand how to alter and apply detection timing parameters in methods and systems of the invention.

### Feedback Aspects

Some embodiments of systems and methods of the invention comprise a feedback controller component that may be configured to make one or more real-time adjustments in one or more samples in the plurality of receptacles. A feedback controller is used in some embodiments of systems and/or methods of the invention as a monitor that receives information about a sample from a sensor, determines whether or not an action is needed with respect to the sample, and triggers one or more actions if deemed necessary. A feedback controller of the invention is configured to utilize ongoing data collection by sensor(s) about one or more samples, and to instigate one or more adjustments in one or more samples in the plurality of receptacles. Non-limiting examples of adjustments are: the receptacle environment, the receptacle temperature, and the receptacle content. For example, though not intended to be limiting, density information is obtained for a sample by detecting a direct detectable parameter in that sample and as a result of that information a feedback controller instigates an action that utilized a fluid-exchange robot of the invention to deliver an inducing agent to one or more samples, thereby adjusting the one or more samples. It will be understood that a detectable parameter identified in a detected sample may instigate one or more alterations in (i) only the detected sample; (ii) one or more samples but not the detected sample, and (iii) the detected sample and one or more additional samples.

A feedback controller included in a system and/or method of the invention permits real-time adjustment of a condition in one or more samples that are in a plurality of receptacles or one or more sets of receptacles. Non-limiting examples of real-time adjustments that can be instigated by a feedback controller in a system and/or method of the invention comprises modifying at least one of a: (a) fluid exchange frequency, (b) fluid exchange volume, (c) sample composition, (d) selection parameter, (f) selection stringency, and (g) selection goal in at least one of the samples in the plurality of receptacles. In some methods of the invention, one or more of samples in a plurality of receptacles comprise a population of bacteriophage and a real-time adjustment comprises at least one of a: (a) fluid exchange frequency, (b) fluid exchange volume, (c) sample composition, (d) selection parameter, (f) selection stringency, and (g) selection goal in at least one of the bacteriophage-containing samples.

### Temperature Control

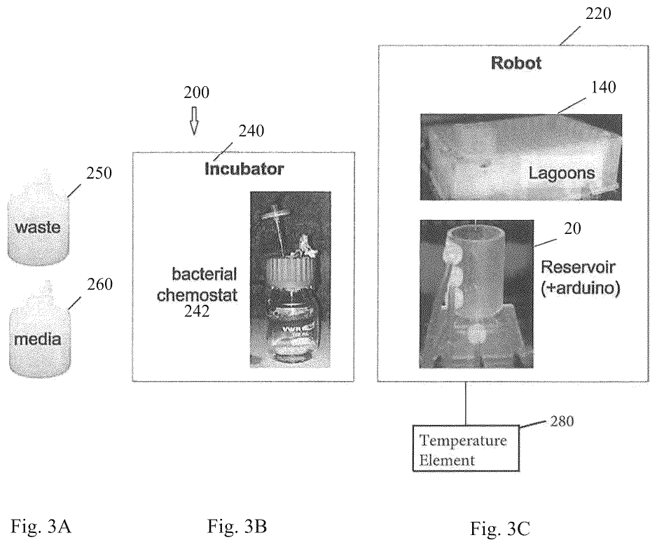

As mentioned above, and as shown in FIG. 3, in one embodiment, the system 200 includes a temperature element 280 configured to selectively modulate a temperature of the samples in the lagoon 140. In one embodiment, the temperature element 280 is associated with the liquid handling robot 220 to provide a heated robot deck. As used herein, the term “deck” means a surface on which one or more components of a robotic system of the invention are placed. In a non-limiting example, components such as a plate reader, one or more reservoirs, one or more receptacles, one or more sets of receptacles, etc. are positioned

on the deck. In some aspects of the invention, a deck may be a temperature-controlled surface. In some embodiments of systems and methods of the invention a deck is enclosed. A deck enclosure may protect components, samples, receptacles, etc. from contamination and may also assist in maintaining a controlled and/or predetermined temperature in certain embodiments of systems and methods of the invention. In one embodiment, the liquid handling robot 120, 220 is equipped with a HEPA hood that prevents contamination from outside bacteria and/or phage. In this particular embodiment, the temperature element 280 for heating the deck of the robot may be configured so that it does not disrupt the HEPA airflow. In one embodiment, the temperature element 280 includes a radiant heater on the interior of the robot enclosure, and one or more smaller heaters above the HEPA vent. According to one embodiment, one of the small heaters is attached to an outlet that is only activated when the deck is below a minimum temperature. In one embodiment, this minimum temperature is approximately 35° C. According to one embodiment, this particular temperature element 280 combination may successfully maintain a constant deck temperature without negatively disrupting airflow. In one embodiment, the temperature element 280 may be configured to regulate the overall temperature of the system 100, 200. In another embodiment, it is contemplated that the temperature element 280 may be configured to selectively modulate the temperature of specific samples in the one or more lagoon receptacles.

In one embodiment, the feedback controller 180 is configured to activate the temperature element 280 in real-time based at least in part on the detectable temperature of one or more samples in the plurality of samples within the lagoon 140. For example, the feedback controller 180 may be configured to activate the temperature element 280 to modulate the temperature in real-time to hold at approximately 37° C. In another embodiment, the feedback controller 180 is configured to activate the temperature element 280 to modulate the temperature in real time to a temperature between one or more of: 25° C. and 40° C., 30° C. and 40° C., 32° C. and 38° C., 34° C. and 37° C., and 34.5° C. and 37.5° C.

### Samples, Cells, Vectors, and Genes

The systems and methods discussed herein may be used to evolve a sample such as a population of bacteriophage. One of ordinary skill in the art will appreciate that in another embodiment, other sample materials and liquid exchange fluids may be used. In one embodiment, the samples may include one or more of molecules such as: genes, RNA, DNA, proteins, enzymes or other molecule of interest to evolve. A sample in a receptacle in a system or method of the invention may also include one or more of a cell, fluid, an inducer, and other components appropriate for PACE methods. Additional general and specific examples of components that may be included in samples are provided elsewhere herein and additional art-known molecules are suitable to include in samples in embodiments of systems and methods of the invention. One of ordinary skill in the art will appreciate that the systems and methods discussed herein may be used to evolve any molecule that can be coded and expressed.

In one embodiment, the detectable parameter (i.e. the parameter detected by the sensor) determines a characteristic of one or more samples in one or more receptacles. The determined characteristic may include at least one of: selection, positive selection, negative selection, biomolecule evolution, temperature, and promoter activity in one or more samples in the plurality of samples.

Cells included in certain embodiments of systems and methods of the invention may be interchangeably referred to herein as “cells” or “host cells.” In certain embodiments of the invention, a host cell is a bacterial cell, a non-limiting example of which is a bacterial cell that can be infected with M13. Continuous evolution procedures in embodiments of systems and methods of the invention comprise F⁺ bacteria that are amenable to M13 infection. As is understood in the art, F⁺ bacteria possess F factor as a plasmid independent of the bacterial genome. A non-limiting example of a host cell that is used in some embodiments of methods and systems of the invention is an E. coli cell. Additional bacterial cells that are amenable to M13 infection are known in the art and are suitable for use in methods and systems of the invention. Additional information about and examples of bacteria suitable for use in methods and systems of the invention are known in the art, see for example, U.S. Pat. No. 9,394,537, the teaching of which is incorporated by reference herein in its entirety.

A host cell included in a system or method of the invention may be a cell comprising a horizontally transferable nucleic acid. In some embodiments, a host cell is provided that comprises at least one viral gene encoding a protein required for the generation of infectious viral particles under the control of a conditional promoter. As a non-limiting example, a host cell may comprise an accessory plasmid comprising a gene required for the generation of infectious phage particles, for example, M13 gIII, wherein the required gene is under the control of a conditional promoter, as described elsewhere herein. In some embodiments of the invention a conditional promoter is inserted into the genome of a host cell, wherein the genome of the host cell also includes a gene required to produce infectious viral particles.

Continued directed evolution systems and methods of the invention may include use of a phage, a viral vector, or naked DNA (e.g., a mobilization plasmid) for delivery or transfer of a gene of interest into a cell, or between cells, respectively. Transfer of a gene of interest from one host cell to a second host cell may be accomplished in a number of ways. For example, though not intended to be limiting, a transfer may occur using a transfer vector that is a virus that infects a cell, such as, but not limited to a bacteriophage or a retroviral vector. In certain embodiments, a viral vector is a phage vector that infects bacterial host cells. In certain embodiments of continuous evolution methods, a transfer vector is a conjugative plasmid transferred from one bacterial cell to a second bacterial cell. It will be understood that in some aspects of the invention, transfer of a gene of interest from one cell to a second cell is dependent on an activity of the gene of interest.

Certain embodiments of systems and methods of the invention utilize horizontally transferable nucleic acids (HTNAs), which are also referred to herein as horizontally transferable genes. HTNAs are genetic material that moves between organisms by means other than by parent to offspring transfer. Non-limiting examples of HTNAs that may be included in some aspects of the invention are bacteriophages, including: but not limited to (i) filamentous phages such as but not limited to: M13/f1/fd; (ii) temperate phages such as but not limited to lambda; and (iii) lytic phages such as but not limited to T1-T7. In certain embodiments of the invention HTNAs are conjunctive plasmids such as, but not limited to: F, F0lac, Col1b and RP4/RK2. It will be understood that other HTNAs can also be used in systems and/or methods of the invention.

In some aspects of the invention one or more HTNAs is secreted by one or more cells in in at least one sample. As a non-limiting example, in certain embodiments of the invention, one or more HTNA is secreted by at least a portion of the cells in one or more of the samples in the plurality of receptacles. In certain embodiments of the invention, a means for secreting a HTNA comprises an export system, such as a DNA export system. A non-limiting example of a DNA export system that may be used in systems and methods of the invention comprise a N. gonorrhoeae DNA export system. In certain aspects of systems and methods of the invention one or more of the secreted nucleic acids are internalized by at least a portion of the cells in one or more of the samples, wherein the internalization means comprises a DNA transporter or other bacterial competence machinery. N. gonorrhoeae export systems, and others suitable for use in embodiments of systems and methods of the invention are known and routinely used in the art, see for example: Hamilton, HL & JP Dillard, Mol Microbiol. 2006 January; 59(2):376-85; and Ibáñez de Aldecoa AL, et al., (2017) Front. Microbiol. 8:1390, doi: 10.3389/fmicb.2017.01390, the content of each of which is incorporated by reference herein in its entirety. In some embodiments of the invention one or more of the samples comprises a population of bacteriophage comprising one or a pIII and pIII* promoter. Non-limiting examples of different components of one or more samples in the plurality of receptacles are: (i) a population of F⁺ bacteria and M13 phage; (ii) a population of bacteriophage and a polymerase; and (iii) a population of bacteria and a polymerase, wherein the polymerase is a T7 RNA polymerase. It will be recognized that other components and combination of components are suitable for use in systems and methods of the invention. Different combinations of components may be selected based on knowledge in the art and routine practices and components associated with traditional PACE methods.

### Detectable Elements and Components

In certain embodiments of the invention a horizontally transferable nucleic acid (HTNA) encodes one or more genes that produce one or more biomolecules, each of which may provide a direct sensor-detectable parameter. The term “detectable elements” as used herein means biomolecules, molecules, and/or agents that can be detected using a sensor of a system or method of the invention. In certain aspects of the invention the detectable element is a biomolecule that is expressed in a cell, for example a cell in a sample. When the one or more biomolecule is expressed in at least one cell in a sample, the biomolecule is directly detectable because of a feature/component of the expressed biomolecule. Non-limiting examples of directly detectable features/components of biomolecules expressed by an HTNA are fluorescent and luminescent moieties. For example, though not intended to be limiting, an expressed biomolecule may comprise one or more of a luminescent moiety and a fluorescent moiety and the moiety's luminescence or fluorescence, respectively, is detectable using a sensor of the invention. Non-limiting examples of fluorescent moieties that may be included in systems and methods of the invention are: mTagBFP2, mTurquoise2, mCeurlean3, EGFP, mWasabi, green fluorescent protein (GFP), SuperfolderGFP, mNeonGreen, mClover3, Venus, Citrine, tdTomato, mRuby3, mScarlet, Fusion Red, mCherry, mStable, and mCardinal. Non-limiting examples of luminescent moieties that may be included in systems and methods of the invention are: luxAB, luxCDE, Fluc, Rluc, and NanoLuc.

Some embodiments of the invention comprise detecting one or more indirect sensor-detectable features/components that result from expression of an HTNA. A non-limiting example of indirect detection in an embodiment of the invention is the production in one or more cells of a sensor-detectable parameter, wherein the production is triggered by the expression of one or more biomolecules encoded by one or more HTNAs. In this situation a HTNA does not encode a directly detectable product, but the expression of the HTNA's encoded product triggers production of second product that is detectable. In a non-limiting example, a biomolecule encoded by a HTNA is expressed in a cell in a sample of the invention; the expressed biomolecule triggers expression of second molecule that comprises a detectable moiety, such as a luminescent or fluorescent moiety; and the moiety's luminescence or fluorescence is detected with a sensor.

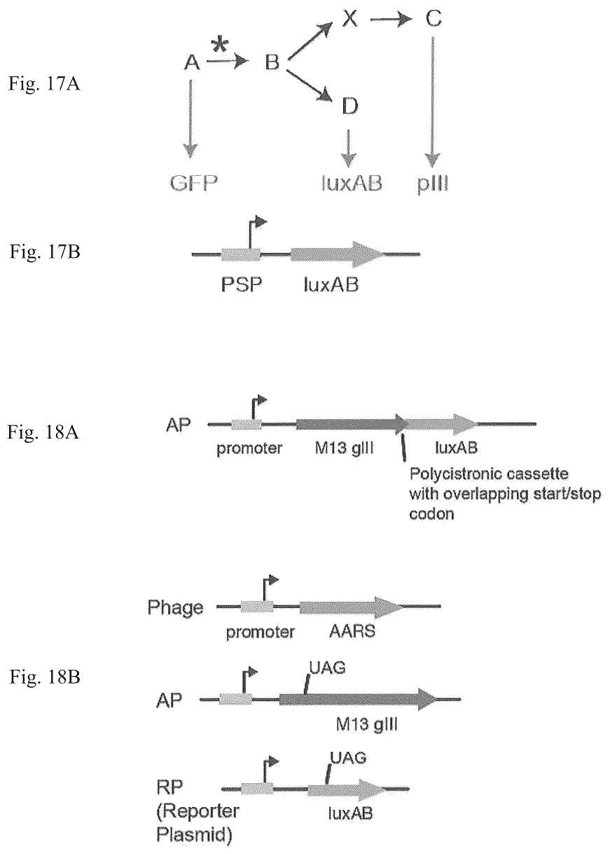

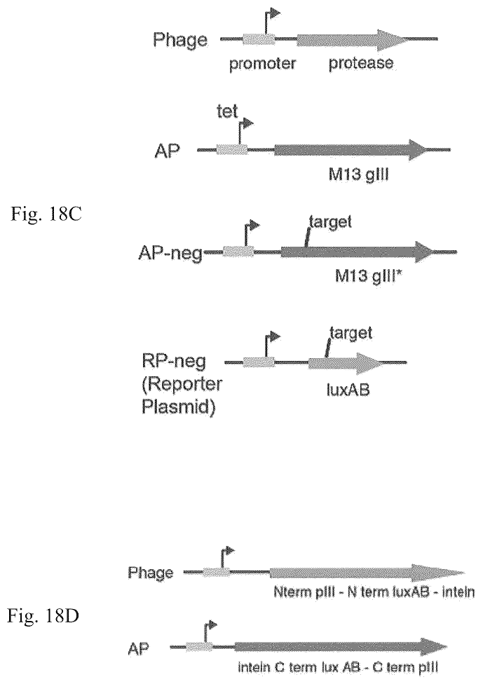

Sensors may be used in embodiments of systems and methods of the invention in monitoring and selection procedures in which the desired biomolecule activity is indirectly tied to pIII production, or for monitoring of other cellular signals. FIG. 17A illustrates a non-limiting example of evolving “enzyme *”, which participates in the biosynthetic pathway shown in FIG. 17A and increases production of X. Readouts of GFP, luxAB, and pIII can be added in separate locations. FIG. 17B illustrates a means for generalized phage titer monitoring, unrelated to selection performance. Additional monitoring and selection procedures are shown in FIG. 18A-D. FIG. 18A shows a selection embodiment in which the desired biomolecule activity results in transcription of pIII. FIG. 18B-D shows selection embodiments in which the desired biomolecule activity results in translation of pIII. FIG. 18B illustrates an embodiment comprising monitoring activity in Trans by enabling readout. FIG. 18C illustrates an embodiment comprising monitoring activity in Trans by disabling readout, and FIG. 18D illustrates an embodiment comprising monitoring activity in Cis.

In some embodiments, systems and methods of the invention can perform phage titer measurements using one or more sensors. For example, though not intended to be limiting, selections where the activity of interest is transduced into transcription of a pIII mRNA can be readily modified to such that they result in a luminescent or fluorescent product that is read by a sensor. If a selection is not amenable to this approach, systems and methods of the invention contemplate additional means. In a non-limiting example of a general mechanism for monitoring the fitness of the population in a continuous directed evolution experimental procedure using a system or method of the invention, a phage titer determination is performed as the experimental procedure progresses.

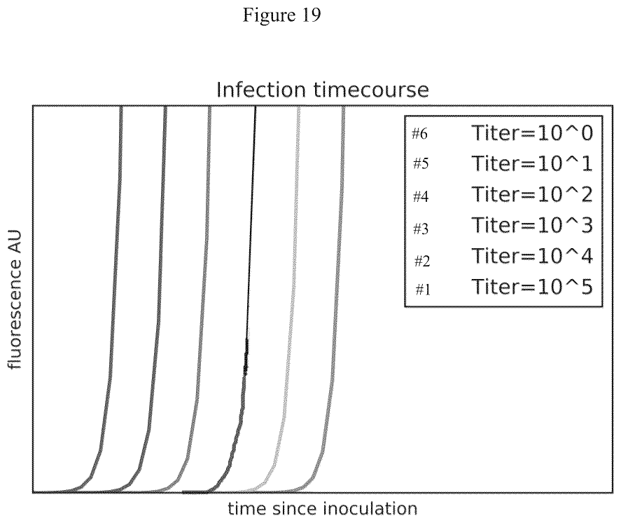

One approach is based on the observation that the “infection curve” that occurs when a lagoon is inoculated with phage depends on the amount of phage in the inoculum. As a non-limiting example, a measurement of phage titer can be achieved by measuring the infection curve of a sample in comparison to a ladder of standard titer phage samples (see FIG. 19). Pipetting operations for these infection curves can be dynamically interleaved with execution of other liquid transfers. Another approach is based on the observation that phage-containing wells exhibit lower absorbance measurements in comparison to no-phage controls, as caused by phage infection slowing the doubling time of the bacteria. As a non-limiting example, a titer estimate can be calculated by comparing the absorbance of the lagoon to no-phage controls as well as the absorbance of the parent chemostat. Previous means used for phage titer measurement comprise use of plaque assays, a technique that is researcher-time intensive and must be incubated overnight. Methods of the invention as disclosed herein are hands free and also provide titer monitoring at shorter timescales than previously possible.

Examples of sensor-detectable parameters that may be detected by sensors in certain embodiments of systems and methods of the invention using one or more of: direct detection and indirect detection of an emission such as, but not limited to: absorbance, luminescence, and fluorescence. Detecting a parameter may comprise determining one or more of a presence, an absence, and an amount of one or more parameter such as absorbance, luminescence, and fluorescence. Other parameters may also be detected in certain embodiments of systems and methods of the invention and additional suitable parameters are known in the art.

Systems and methods of the invention permit flexibility in detection strategies. For example, detection can be independently performed on different samples in the system and/or method of the invention, which permits detection of one sample, a portion of all samples, or all samples in a continuous evolution procedure. A system of the invention may also be configured such that each sample is detected using an independently selected means, time, and schedule of detection. Detection means and timing and their application in some embodiments of systems and methods of the invention are described in more detail elsewhere herein.

Provided herein are systems and methods that include one or both of direct and indirect detection of a parameter in one or more cells in a sample. Indirect and direct detection means can be used in systems and methods of the invention to identify in a sample one or more characteristics of the sample. Characteristics of cells, cell populations, and samples can be determined using certain embodiments of systems and methods of the invention. Numerous determinable characteristics are known and routinely assessed in the art and such determinations are routinely carried out as part of continuous evolution methods.

Non-limiting examples of characteristics determinable using methods and systems of the invention include: selection, positive selection, and negative selection, which are characteristics of cells, cell samples, cell populations, organism populations that are routinely determined in the art. As used herein, the term “selection” is used in reference to the inclusion of a gene of interest in a sample comprising cells. The term “positive selection” is used herein in reference to positive, directional selection in which an advantageous and/or desirable gene is at an increased frequency in a sample's cell population. The term “negative selection” is used herein in reference to negative, directional selection in which a deleterious and/or undesirable gene is reduced in frequency in a sample's cell population. Additional non-limiting examples of characteristics in cells and samples that can be determined using embodiments of systems and methods of the invention are: enzyme activity; phage population size; bacterial population size; presence, absence, and/or amount of gene transcription; and presence, absence, type, and/or amount of biomolecule evolution.

### Additional Terms

As used herein the term “plurality” used herein in reference to an item, such as but not limited to: cells, samples, and receptacles, is always more than one, and in certain embodiments of the invention can be one or more of: at least 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 or more cells, samples, or receptacles, respectively, including every integer between each listed number. As non-limiting examples, in some embodiments of the invention a plurality of samples is one or more of: at least 3 samples, at least 5 samples; at least 10 samples; at least 15 samples; at least 50 samples; at least 100 samples; at least 500 samples; at least 1000 samples; and at least 10,000 samples. As non-limiting examples, in some embodiments of the invention a plurality of cells is one or more of: at least 3 cells; at least 5 cells; at least 10 cells; at least 15 cells; at least 50 cells; at least 100 cells; at least 500 cells; at least 1000 cells; and at least 10,000 cells.

As used herein the term “portion” means “part of” when used in reference to items and sets of items, such as but not limited to: cells, samples, and receptacles. Thus, as used herein, the term “at least a portion” means at least one, and in some embodiments means one to all of the items referenced. As a non-limiting example, at least a portion of cells in a sample means at least one of the cells, which in some embodiments of the invention means one or more of: at least 1, 2, 3, 4, 5, 6, 7, 8, 9, 10, 11, 12, 13, 14, 15, 16, 17, 18, 19, 20, 25, 30, 40, 50, 60, 70, 80, 90, 100, up through all of the cells in the sample.

As used herein, the terms “nucleic acid” and “polynucleotide” are used interchangeably, and understood to include one or more of: natural nucleosides, nucleoside analogs, and other variant nucleosides many of which are known and routinely used in the art. The terms “polypeptide” and “protein” are used interchangeably herein and may refer to

one or more of: protein complexes, natural polypeptides, synthetic polypeptides, and recombinant polypeptides. Polypeptides used in embodiments the invention may comprise one or more of: a natural amino acid, a non-natural amino acid, an amino acid analog, and other variants. Structure, function, and use of variants of polypeptides are well known in the art. See for example, U.S. Pat. No. 9,394,537, which is incorporated by reference herein in its entirety.

The term “lagoon,” is used interchangeably with the term “receptacle” and when used in systems and methods of the invention, it refers to the container that typically holds a sample and components used in continuous directed evolution methods. The lagoon or receptacle has a “flow” of components such as cells, inducing agents, fluids, etc., which may be performed by the liquid handling robot. In certain methods of the invention a lagoon may hold a population of host cells and a population of viral vectors replicating within the host cell population. A liquid handling robot may be used in conjunction with one or more lagoons of the invention in certain methods of the invention for actions such as, but not limited to: adding, mixing, and removing components, as described elsewhere herein.

The term “cellstat,” as used herein, refers to a culture vessel comprising host cells, in which the number of cells is substantially constant over time.

The terms “function” and “activity” of a gene of interest, are used interchangeably herein in reference to a function or activity of a gene product, for example, a nucleic acid, or a protein, that is encoded by the gene of interest. As non-limiting examples, a function of a gene of interest may be an enzymatic activity, an ability to activate transcription targeted to a specific promoter sequence, a bond-forming activity, and a binding activity such as a protein, DNA, or RNA binding activity.

The term “promoter” is used herein in reference to a nucleic acid molecule with a sequence recognized by the cellular transcription machinery and that is capable of initiating transcription of a downstream gene. A promoter may be constitutively active, meaning that the promoter is always active in a given cellular context, or it may be conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A non-limiting example of a conditionally active promoter is an inducible promoter that is active only in the presence of an “inducer” or “inducing agent”. A non-limiting example of an inducible promoter is an arabinose-inducible promoter. A variety of constitutive, conditional, and inducible promoters are well known and routinely used in the art and one skilled in the art will be able to identify and use suitable promoters, including inducible promoters in embodiments of systems and methods of the invention.

### General System Use and Methods

FIG. 2 provides a schematic view of one embodiment of a system 100 for conducting continuous directed evolution. In this illustrative embodiment, the system 100 includes receptacles configured to hold a plurality of samples. As illustrated, the plurality of receptacles 142, where each receptacle 142 is configured to contain a sample. As set forth in more detail elsewhere herein, the plurality of receptacles 142 are configured for liquid exchange (i.e. the system 100 is configured to selectively add and/or remove liquids from the receptacles). As shown in FIG. 2, the system 100 further includes a liquid handling robot 120 configured to perform liquid exchange in one or more receptacles 142 in the plurality of receptacles. In other words, the liquid handling robot 120 is configured to selectively dispense one or more liquids into one or more of the receptacles 142, selectively mix the liquid in one or more of the receptacles, and/or selectively remove liquids from one or more of the receptacles 142.

In the embodiment illustrated in FIG. 2, the system 100 also includes a sensor configured to detect at least one parameter of one or more samples in the plurality of samples. In one embodiment, the sensor is configured to detect at least one parameter of one or more samples while the one or more samples remain within the receptacles 142. In another embodiment, the detecting may be performed while the sample is outside of the receptacle. For example, in one illustrative embodiment, the sensor is configured as an integrated plate

reader 160. The integrated plate reader 160 may include a plate 162, and the liquid handling robot 120 may be configured to transfer an aliquot of the one or more samples from the receptacles 142 into a receptacle in another plate, which is also referred to herein as a “detection plate”. The integrated plate reader 160, or a separate non-integrated plate reader may be configured to detect at least one parameter of the aliquot after it has been transferred to the different receptacle. The detection plate 162 may have a plurality of detection receptacles configured to contain one or more samples. Further details of these components of the system 100 are discussed in greater detail elsewhere herein.

In one illustrative embodiment, the system 100 also includes a feedback controller 180 configured to make at least one real-time adjustment in one or more samples in the plurality of samples based upon the at least one detectable parameter. Further details of the feedback controller 180 are set forth below. As shown in FIG. 2, and as discussed in more detail below, the system 100 may further include a reservoir 20 configured to hold an independently selected liquid exchange fluid. Various liquid exchange fluids are discussed in more detail below, but may for example include a culture, such as a bacteriophage solution. Also, as shown in FIG. 2, in one embodiment, the system 100 may include one or more additional liquid exchange fluids, such as inducing agents, to be selectively added to one or more of the samples.

In one illustrative embodiment, a plurality of receptacles is wells in a 96-well plate. In another embodiment, the receptacles may include a plurality of well plates, and it is also contemplated that the plurality of well plates may be stacked on top of each other, which conserves space. In one embodiment, the system comprises at least 1, 5, 10, 15, 20, 30, 40, 50, 60, 70, 80, 90, 100, 150, 200, 250, 300, 350, 400, 450, 500, 550, 600, 650, 700, 750, 800, 850, 900, 950, 1000 or more receptacles. In another embodiment, the system comprises at least 1, 2, 3, 4, 5, 6, 7, 8, 9, 10, 15, 20, 25, or more sets of receptacles. It is contemplated that the plurality of receptacles 140 may include an independently selected number of receptacles 142. Certain embodiments of the invention, may, for example, include 96, 192, or 384 receptacles. It is envisioned that certain embodiments of systems and methods of the invention may include 1, 2, 3, 4, 5, 6, 7, 8, 9, 10, or more sets of 96, 192, 384, or other number of receptacles.

The liquid handling robot 120 may include an arm which, in one particular embodiment, is configured to deliver fresh bacteria to each phage population, remove waste, and add customized amounts of liquid exchange fluids, such as inducers, to control experimental conditions. The liquid handling robot 120 may be configured to simultaneously perform hundreds of parallel PACE experiments.

As shown in FIG. 2, the liquid handling robot 120 may include a plurality of tips 122 configured to selectively dispense and/or transfer an independently selected liquid exchange fluid into and out of one or more of the receptacles 142. In one embodiment, it is contemplated that the liquid handling robot 120 is configured to dispense a plurality of samples into a plurality of receptacles 142. In another embodiment, a plurality of samples may already be provided within the receptacles 142 and the liquid handling robot 120 may be configured to perform liquid exchange on the plurality of samples. As mentioned above, it is contemplated that the liquid handling robot 120 may be configured to transfer an aliquot of one or more samples out from the receptacle 142 and into a detection receptacle 140. A sensor 160 may be configured to detect one or more parameters of the aliquot while the sample aliquot is in the detection receptacle. As mentioned above, in one embodiment, the integrated plate reader may include a plate 162 which may have a plurality of detection receptacles configured to contain one or more samples.

The inventors of the present disclosure recognized that in conventional systems for conducting PACE, researchers typically use a luminescence readout to measure the fitness of phage populations produced by PACE. However, this assay is performed using a separate, manually isolated sample due to the difficulty of automatically monitoring populations maintained using peristaltic pumps. In contrast, as discussed in greater detail elsewhere herein, aspects of the present disclosure are directed to a system for conducting PACE that includes a sensor, such as an integrated plate reader 160, which may be configured to monitor at least one parameter of the one or more evolving population samples in real time within one or more of the plurality of receptacles 140. As set forth in more detail elsewhere herein, the one or more parameters may include one or more of absorbance, luminescence, and fluorescence, etc. In one embodiment, the system may be configured such that the data can be integrated into a feedback-and control system, through feedback controller 180, e.g. data that enters the feedback and control system may trigger actions by the system to increase the mutation rate in experiments that have had static fitness for a long period of time, or decreasing the flow-through rate populations that would otherwise wash out.

Turning now to FIG. 3, a schematic view of one embodiment of a system 200 for conducting PACE is illustrated. In this illustrative embodiment, the system 200 includes one or more pluralities of receptacles 140 configured to hold a plurality of samples, a liquid handling robot 220 configured to perform liquid exchange in one or more receptacles, and a reservoir 20 configured to hold an independently selected liquid exchange fluid.

As shown in FIG. 3, in one illustrative embodiment, the independently selected liquid exchange fluid may include bacterial chemostat 242. As used herein, the term “chemostat” means a device in which bacteria are kept suspended in a culture medium that is continuously renewed by continuous flow of new medium through the device. In one embodiment, it is an off-deck bacterial chemostat 242 that may be housed within an incubator 240 and may be selectively serviced by peristaltic pumps. In one embodiment, this chemostat is kept at approximately 37° C. in the incubator 240. Other desired temperature ranges are discussed in more detail below. In one embodiment, the chemostat 242 is stirred with a stir bar. Separate media containers (also referred to as carboys) 260 and waste containers (carboys) 250 may be used to manage liquid for this component. As shown, in one embodiment, the system 200 further includes a temperature element 280 configured to selectively modulate a temperature of one or more of the samples in the plurality of receptacles. Furthermore, as shown in FIG. 3, in one embodiment the reservoir 20 is an on-deck reservoir configured to hold the independently selected liquid exchange fluid. In one embodiment, the independently selected liquid exchange fluid is the sample material, and may, for example be a bacterial culture. Other liquid exchange fluids are discussed below.

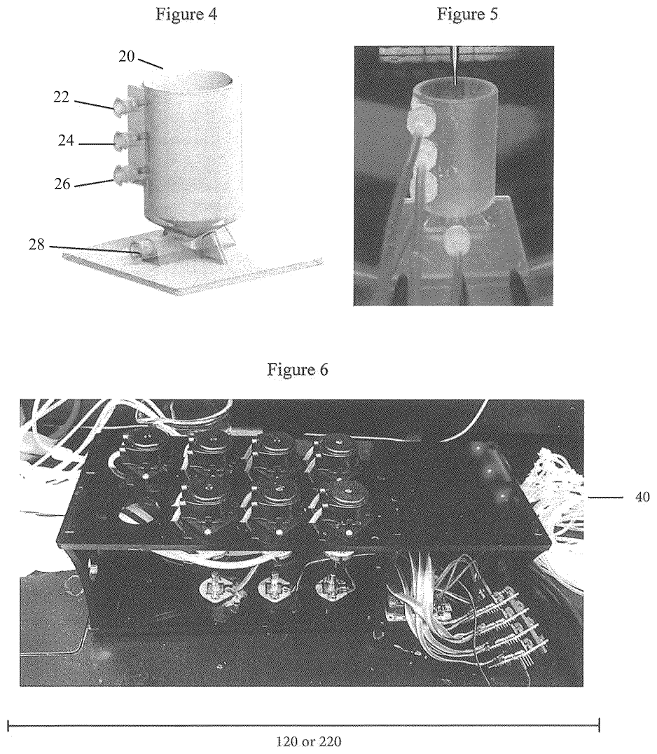

FIGS. 4 and 5 illustrate a schematic view of certain embodiments of a reservoir 20 configured to hold an independently selected liquid exchange fluid. In one embodiment, a plurality of reservoirs 20 may be provided. In another embodiment, one reservoir 20 may be provided and it may be used to selectively hold a plurality of liquid exchange fluids which are utilized during liquid exchange with the liquid handling robot.

In one illustrative embodiment, the reservoir 20 includes a plurality of ports 22, 24, 26, 28, and as shown in FIG. 5, tubing connects each port with its upstream and/or downstream location as would be readily understood by one of ordinary skill in the art. One or more of these ports may enable the liquid handling robot 120, 220 to access the independently selected liquid exchange fluid. In one embodiment, the ports 22, 24, 26, 28 may be used to 1) fill the reservoir 20 with water, 2) fill the reservoir 20 with a cleaning solution, 3) fill the reservoir with the sample material, and then also to 4) selectively drain the reservoir 20 of any of the above liquid exchange fluids.

In one embodiment, the independently selected liquid exchange fluid includes a bacteriophage solution. In one embodiment, the liquid handling robot 120, 220 is configured to selectively dispense the bacteriophage solution into one or more receptacles in the plurality of receptacles. It is also contemplated that the independently selected liquid exchange fluid may include an inducing agent. In the particular embodiment illustrated in FIGS. 4 and 5, the reservoir 20 has at least four ports including a first port 22 to fluidly couple the reservoir to a water line, a second port 24 to fluidly couple the reservoir to a cleaning fluid line, a third port 26 to fluidly couple the reservoir to a liquid exchange fluid line, and a fourth port 28 to fluidly couple the reservoir to a drain line.

In one illustrative embodiment, the upper three ports 22, 24, 26 are used to fill the reservoir, while the fourth port 28 is the drain. In one embodiment the ports 22, 24, 26, 28 are leur-lock ports, however, one of ordinary skill in the art would recognize that other types of ports may also be utilized as the present disclosure is not limited in this respect. In one embodiment, the reservoir 20 may be manufactured via 3D printing. In one particular embodiment, the reservoir 20 may be manufactured via Form 2 resin based 3D printing.

Aspects of the present disclosure are directed to systems and methods for conducting PACE which may include one or more features directed at preventing cross-contamination of the samples. For example, in one embodiment, over the course of running the system, after the liquid handling robot 120, 220 services the receptacles 140 and provides one or more liquid exchange fluids (i.e. bacterial culture sample), the reservoir 20 is cleansed. In one illustrative embodiment, the cleaning process includes draining the reservoir 20, filling with a cleaning solution, draining the cleaning solution, and thereafter rinsing one or more times with water. After the cleaning process, the reservoir is then filled with a fresh sample (i.e. fresh bacterial culture). A schedule of cleaning process can be set by a user. For example, a cleaning process may be performed after the liquid handling robot services a plurality of samples with a fluid, and in certain instances, a cleaning process is performed after delivery of a first fluid and prior to delivery of a subsequent fluid. It will be understood how to schedule and time the cleaning process to prevent cross-contamination of samples.

In one embodiment, the cleaning solution is a 10% bleach and 90% water solution. However, one of ordinary skill in the art would recognize that other cleaning solutions, including other bleach concentrations or other suitable disinfecting solutions may also be used as the present disclosure is not so limited. Furthermore, in one embodiment, after the cleaning solution is drained from the reservoir, the reservoir is rinsed at least three times with water before the reservoir 20 is filled with a fresh sample. One of ordinary skill in the art would appreciate that the specific cleaning cycle, and number of rinse cycles may vary.

The inventors recognized that in certain embodiments, the cleanse cycle is essential to prevent cross-contamination of phage. Furthermore, the inventors recognized that in certain embodiments, it is critical that the bacterial culture is fresh, as a well-agitated bacterial culture in the correct growth phage may be essential for PACE. In one embodiment, it is desirable to have a system for conducting PACE that is capable of running as many parallel PACE experiments as possible without having cross-contamination of the samples.

Turning now to FIG. 6, a portion of a liquid handling robot 120, 220 is shown in greater detail. In this illustrative embodiment, the liquid handling robot 120, 220 includes a plurality of pumps 40. In one embodiment, the liquid handling robot 120, 220 includes a bank of raspberry pi driven pumps 40. In one embodiment, the liquid handling robot 120, 220 includes software, such as Hamilton robot software which is configured to trigger filling and/or draining of the reservoir by, for example, calling . bat files that interface with a bank of a plurality of pumps 40. In one illustrative embodiment, there are seven pumps 40, but in other embodiments, there may be up to eight or more pumps 40 that are driven by a raspberry pi. One primary design consideration of this component may be robustness. Additionally, in one embodiment, the liquid handling robot 120, 200 includes peristaltic pumps, which may be desirable as they are a relatively economical type of pump. Furthermore, in one embodiment, the system includes encoders that can be used to calibrate pump rate and/or correct for any aging pumps. In one embodiment, the liquid handling robot 120, 220 comprises one or more peristaltic pumps configured to selectively perform liquid exchange in one or more samples in the plurality of samples in the lagoon 140. In one particular embodiment, each sample in the plurality of samples is contained in a separate receptacle 142. It will be understood that other types of suitable pumps may be used in embodiments of systems and methods of the invention

As mentioned above, the sensor 160 may be configured to detect at least one parameter of one or more samples. In one embodiment, the sensor 160 is configured to detect at least one parameter of at least a portion or all of the plurality of samples within the receptacles. Furthermore, as mentioned above, the feedback controller may be configured to make at least one real-time adjustment in one or more samples in the plurality of samples based upon the at least one detectable parameter. In another embodiment, the feedback controller is configured to make at least one real-time adjustment in all of the plurality of samples based upon the at least one detectable parameter.

Furthermore, as mentioned above, the sensor may be configured to detect at least one parameter of the one or more samples while the one or more samples are in the receptacle 140. In another embodiment, the detecting may occur outside of the receptacle 140. For example, the liquid handling robot may be configured to transfer an aliquot of the one or more samples in the plurality of samples from the receptacle 140 into a detection receptacle, and the sensor may be configured to detect at least one parameter of the aliquot while the aliquot is in the detection receptacle. As mentioned above, in the embodiment shown in FIG. 2, the integrated plate reader includes a plate 162 having a plurality of detection receptacles.

Aspects of the present disclosure are directed to a system for conducting continuous directed evolution that includes a sensor configured to detect at least one detectable parameter of one or more of samples within the receptacles. In one illustrative embodiment, the sensor is an integrated plate reader 160, which may, as a non-limiting example be a ClarioSTAR plate reader (BMG Labtech, Cary, NC) or an Infinite 200 PRO (Tecan Group Ltd, Männedorf, Switzerland). As described elsewhere herein, the detectable parameter may include one or more of absorbance, luminescence, fluorescence, etc. In one embodiment, the sensor is configured to detect one of more of a presence, an absence, and/or an amount of the detectable parameter.

An embodiment of a system or method of the invention may also include determining a characteristic of one or more of the samples, based at least in part on one or more detectable parameters. A determined characteristic may include at least one of: selection, positive selection, negative selection, an enzyme activity, phage population size, gene transcription, biomolecule evolution, and promoter activity in the one or more samples. Furthermore, the detecting may include detecting one of more of a presence, an absence, and an amount of the detectable parameter. The detectable parameter may include one of more of absorbance, luminescence, and fluorescence. According to one embodiment, one or more samples in the plurality of samples are modified in real-time based upon at least one of the detected absorbance, luminescence, and fluorescence.

According to one embodiment, one or more of the samples include a bacteriophage population, and the modification of one or more samples in the plurality of samples is

selected for one or more of: (a) modifying a positive selection strength of at least one bacteriophage sample in response to the detectable parameter; (b) modifying a negative selection strength of at least one bacteriophage sample in response to the detectable parameter; (c) modifying a mutation rate of at least one bacteriophage sample in response to the detectable parameter; and (d) modifying a selection goal of at least one bacteriophage sample in response to the detectable parameter. A non-limiting example of an action that may be used to modify one or more samples in the plurality of samples is an addition of an inducing agent to one or more samples in the plurality of receptacles in response to the detectable parameter.

A method for conducting continuous directed evolution according to the invention may also include detecting a second parameter in one or more of the samples in the plurality of samples, and modifying one or more samples in the plurality of samples in real-time based at least in part on the second detected parameter of the one or more samples. The method may further include providing one or more reservoirs, wherein each reservoir is configured to hold and selectively dispense an independently selected liquid exchange fluid, sterilizing one or more of the reservoirs with a solution comprising bleach, and placing one or more of the independently selected liquid exchange fluids into a selected reservoir of the one or more reservoirs. In one embodiment, a system and/or method of the invention includes one or more of: an independently selected liquid exchange fluid comprising a bacterial culture, an independently selected liquid exchange fluid comprising a bacteriophage solution, and an independently selected liquid exchange fluid comprising an inducing agent.

It is contemplated that in one embodiment, at least one of the one or more reservoirs 20 has at least four ports including a first port 22 to fluidly couple the reservoir to a water line, a second port 24 to fluidly couple the reservoir to a cleaning fluid line, a third port 26 to fluidly couple the reservoir to a liquid exchange fluid line, and a fourth port 28 to fluidly couple the reservoir to a drain line. The liquid handling robot 120, 220 may further include one or more peristaltic pumps configured to selectively perform liquid exchange in one or more samples in the lagoon 140. Each sample may be contained in a separate receptacle, and the lagoon 140 may include a plurality of samples.

As mentioned above, the method for conducting continuous directed evolution method, such as PACE methods, simultaneously on a plurality of samples may include a sterilizing step. According to one embodiment, the sterilizing step includes washing the reservoir with the solution comprising bleach, where the solution comprises at least 8%, 9%, 10%, 11%, or 12% bleach, and rinsing the reservoir with water. In one embodiment, the solution comprises 10% bleach. In one embodiment, the rinsing step is performed at least 2, 3, 4 5, 6, 7, 8, 9, 10 or more times. In one embodiment, the detecting step includes detecting at least a first parameter of each of the samples. Furthermore, the detecting step may include transferring an aliquot of the sample from the receptacle 142 containing the sample into a detection receptacle, and detecting the at least one parameter of the aliquot while the aliquot is in the detection receptacle. Also, the modifying step may include modifying each of the samples within the lagoon in real-time based upon the first detectable parameter.

In some embodiments of the invention, one or more of samples in a plurality of samples comprises a population of bacteriophage and the determined characteristic comprises a level of one or both of pIII and pIII* transcription in one or more of the samples in the plurality of receptacles in the system. As is recognized in the art, for a continuous evolution circuit, (for example a PACE circuit), the circuit must trigger production of an essential phage gene. In some aspects of the invention, a phage gene that is triggered is one or a pIII gene (also referred to as a p3 gene) and a pVI gene. In certain embodiments of systems and methods of the invention, other determined characteristics can be monitored, for example monitoring production of a different component of the selection circuit. For example, if one is evolving a metabolic pathway, one may want to monitor how the bacteria up-regulate some other enzyme (which may not necessarily contribute to pIII production) in response to entry of the evolving protein to the cell. In another embodiment, one may monitor gene activity directly by disabling a readout. For example, it may include protease both cut a linker in pIII* and also the fluorophore side-by-side. In another embodiment, one may monitor a proxy for phage population size. By placing a readout protein under control of the Phage Shock Promoter (PSP), one may get a readout of total phage population, see for example FIG. 17B. Additionally, one may have the selection directly activate a readout, see for example FIG. 17A. For example, it is also contemplated that one could evolve intron/intein splicing, by placing a luciferase in a linker region of pIII and challenging the phage to repair a splice in the middle of the luciferase: this would turn on luminescence and active pIII at the same time. FIGS. 17A-B and 18A-D provide examples of various evolution strategies that can be carried out using certain embodiments of the invention.

In certain embodiments of systems and methods of the invention, one or more samples in a plurality of samples comprise a population of F⁺ bacteria and M13 phage. In another embodiment, one or more samples in the plurality of samples include a population of bacteriophage and a polymerase. In one particular embodiment, the polymerase is a T7 RNA polymerase. The system for conducting PACE may be configured such that in one embodiment, the sensor is configured to detect over a time period of at least 1, 3, 6, 12, 18, 24, 36, 48, or more hours. In one embodiment, the sensor is configured to detect continuously over the time period.

The system may be configured such that the real-time adjustment includes adjusting a condition in one or more samples in the plurality of samples. In one embodiment, the real-time adjustment includes modifying at least one of a: (a) fluid exchange frequency, (b) fluid exchange volume, (c) sample composition, (d) selection parameter, (f) selection stringency, and (g) selection goal in at least one sample in the plurality of samples.

In a further embodiment, one or more samples in the plurality of samples includes a population of bacteriophage and the real-time adjustment comprises at least one of a: (a) fluid exchange frequency, (b) fluid exchange volume, (c) sample composition, (d) selection parameter, (f) selection stringency, and (g) selection goal in at least one of the bacteriophage-containing samples.

In another embodiment, one or more samples includes a population of bacteriophage, and the feedback controller is configured to adjust a positive selection strength of at least one of the bacteriophage-containing samples in response to the at least one detectable parameter.

In another embodiment, one or more samples includes a population of bacteriophage, and the feedback controller is configured to adjust a negative selection strength of at least one bacteriophage-containing sample in response to the at least one detectable parameter.

In yet another embodiment, one or more samples in the plurality of samples comprises a population of bacteriophage, and the feedback controller is configured to adjust a bacteriophage mutation rate in at least one bacteriophage-containing sample in response to the at least one detectable parameter. In yet another embodiment, one or more samples includes a population of bacteriophage, and the feedback controller is configured to adjust a selection goal of at least one bacteriophage-containing sample in response to one or more of the at least one detectable parameter.

In one embodiment, a system for conducting PACE may be used for real-time monitoring and it may be configured to enact one or more of the following modifications to experiments on an individual/bulk basis: 1) flow-through rate in one or more receptacles; 2) chemostat flow-through rate; 3) addition of inducer chemicals (non-limiting examples of which are for positive and negative selection strength) to receptacles and/or chemostats; 4) receptacle and/or chemostat volume (changes the population size); 5) selection goal(s) for a lagoon, including modulating the relative proportion of multiple bacteria in a lagoon; and 6) evolving optogenetic properties of proteins by having the robot control the color and intensity of different colors of light that the lagoons are exposed to. Furthermore, it is also contemplated that global parameters may also be modified including deck temperature; and if using a single off-deck chemostat, flow-through rate of that chemostat. It is also contemplated that the system for conducting PACE discussed herein may be used to automatically seed new PACE experiments by splitting successful receptacles. This may be useful in the scenario where one is searching for appropriate conditions for a particular selection, and one wants to have the liquid handling robot identify which conditions were best and go ahead and try more similar conditions.

In another exemplary embodiment, the sensor 160 is configured to detect at least a first detectable parameter and a second detectable parameter in one or more samples in the plurality of samples within the lagoon, and the feedback controller 180 is configured to make one or more real-time adjustments in the one or more of the plurality of samples based at least in part on the first detectable parameter and the second detectable parameter. In one embodiment, the means of detecting the first and second detectable parameters comprises detecting a first and a second detectable agent, respectively. The first detectable agent may include a first fluorescent molecule and the second detectable agent may include a second fluorescent molecule. One or more samples in the plurality of samples may include a population of bacteriophage and/or a polymerase, and in one particular embodiment, the polymerase includes a T7 RNA polymerase.

In a non-limiting example, a first detectable agent includes a mCherry molecule and its presence in the sample indicates the first characteristic of positive selection in the sample and a second detectable agent comprises a GFP molecule and its presence indicates the second characteristic of negative selection in the sample. Furthermore, the feedback controller may be configured to adjust one or more of the samples in the plurality of samples in real time, based at least in part on the determination of at least one of the first characteristic and the second characteristic. The first detectable agent may include a first luminescent molecule and the second detectable agent may include a second luminescent molecule. Also, the feedback controller may be configured to adjust the plurality of samples in real time by directing the liquid handling robot to add a liquid exchange fluid, such as an inducing agent, to one or more samples in the plurality of samples. It is contemplated that the inducing agent may include at least one of L-arabinose, anhydrotetracycline, and theophylline-sensitive riboswitch, or another suitable agent as disclosed elsewhere herein or known in the art.

As described elsewhere herein, the transcription of an essential phage gene may be determined directly or indirectly using a system and/or method of the invention. A non-limiting example of an indirect detection means may include detecting production of a different component of the selection circuit. For example, when evolving a metabolic pathway, systems and methods of the invention can be used to detect up regulation by the bacteria of another enzyme, which may, but need not necessarily contribute to pIII production, wherein the up regulation is in response to entry of the evolving protein to the cell.

As described elsewhere herein, in certain embodiments of the invention include a selection phage, the genome of which is deficient in at least one gene that is required for the generation of infectious phage particles and a gene of interest to be evolved using a system or method of the invention. In a non-limiting example, an M13 selection phage may comprise a genome capable of all other phage functions required for the phage life cycle (for example include a gI, gII, gIV, gV, gVI, gVII, gVIII, gIX, and gX gene) but be defective in that it does not include a full-length gIII gene, and thus be unable to generate infectious phage particles. It will be understood that a gene of interest may be included in the genome of a selection phage that is included in an embodiment of the invention.

In a non-limiting example, certain embodiments of systems, components, and methods of the invention include a viral vector, for example, a selection phage, and a matching accessory plasmid. Certain embodiments of the invention include a vector system for phage-based continuous directed evolution that comprises (i) a selection phage deficient in a gene that is necessary to produce infectious phage particle, and that comprises a gene of interest to be evolved; and (ii) an accessory plasmid that comprises the gene that is necessary to produce infectious phage particle, and that is under the control of a conditional promoter, such that the conditional promoter is activated by a function of a gene product encoded by the gene of interest. In addition, some embodiments of systems and methods of the invention may include a vector system that additionally comprises a helper phage. In this situation, a selection phage may not include all genes required for the generation of phage particles, and the genome of the selection phage is complemented by the helper phage genome such that the helper phage genome and the selection phage genome together make up at least one functional copy of all genes necessary to produce phage particles, but are deficient in at least one gene required for the generation of infectious phage particles. In an alternative non-limiting example, a vector system may include an accessory plasmid that includes an expression cassette that includes the gene necessary to produce infectious phage. In some embodiments of the invention, the accessory plasmid is under the control of a conditional promoter and the activity of the conditional promoter is dependent on activity of a produce of a gene of interest.

It will be understood that other arrangements and combinations of vectors, gene deficiencies, accessory plasmids, mutagenesis plasmids, helper plasmids, helper plasmids that include expression constructs of one or more genes that are not present in the selection phage genome or in the accessory plasmid, conditional promoters, etc. can be included in embodiments of systems and methods of the invention. Additional information is available at least in U.S. Pat. Nos. 9,394,537 and 9,771,574, the teaching of each of which is incorporated by reference herein in its entirety.

### Kits

It is contemplated that the invention encompasses full systems, partial systems, and components of a system of the invention. Thus, in some aspects of the invention, kits provided may include one or more components of a system as described herein. In a non-limiting example a kit may include one or more of a liquid handling robot, a detector, a sensor, a reservoir, a temperature controller, and a feedback controller. A kit may also include instructions for use of one or more components to perform a method of the invention and/or for use in a system of the invention. Certain embodiments of kits for use in systems and methods of the invention comprise one or more solutions, bacteria strains, cultures, enhancers, horizontally transferrable nucleic acids, vectors, media, culture fluid, cleaning fluid, and wash fluid. A kit of the invention may include all, or a portion of the components of a system of the invention. Elements in a kit may, in some embodiments, be used in conjunction with components in a system of the invention and/or with other components that are suitable for use in methods of the invention.

### Examples

### Example 1

Discretized continuous directed evolution experiments were performed and details components and procedures are provided. The specific physical configuration of the robot is flexible and can be varied, which permitted optimization of the set up for particular experiment. Examples of parameters of continuous directed evolution that are run using embodiments of the system: running the maximum number of simultaneous experiments; having each experiment be fed by a different type of bacterial strain; running each experiment with different conditions having the parameters of one or more experiments vary in response to plate reader measurements; having the robot be self-sufficient, and not require the researcher to add more consumables, for the maximum amount of time. The flexibility of the system permitted different parameters and conditions to be utilized in continuous directed evolution.

### Materials

An image of an embodiment of a system of the invention is shown in FIG. 15. Parts included in that system are described below. A liquid handling robot, Hamilton STARlet, (Hamilton Robots, Reno, NV) equipped with:
Integrated plate reader, BMG Labtech CLARIOstar (BMG Labtec, Cary, NC)
iSWAP, a device can pick up, move, and put down plates (Hamilton Robots, Reno, NV)
HEPA Hood to Prevent Contamination
96-bulk-addressable head
Individually-addressable 8-channel head
Shaker plates (Big Bear Automation, Freemont, CA)
Standard incubator capable of maintaining 37° C.
Standard magnetic stir plate
Peristaltic pumps, such as from the MasterFlex series (Cole Palmer, Vernon Hills, IL)]
Deep dish 96-well plate, such as racks of library tubes (VWR, Radner, PA]
Plate reader plates (Corning Life Sciences, Corning, NY) Chemicals used as inducers
L-arabinose, Theophylline, Glucose, Mutagenic Nucleoside (dp) Partial List Additional parts:
Components for tubing/leur locks/containers, etc.
Male Luer for Tubing, various sizes; Female Luer for Tubing, various sizes; T/Y Connections, various sizes; Luer adapter Tee, female x female x female; Barbed-Y connector, various sizes; Luer Plug/Caps; Female Luer Cap; Male Luer Lock Plug (Cole Palmer)

### Robotic Pace

Experiments using robotic PACE systems and methods were performed. The number of receptacles used was 10, 14, or 16 depending on the experiment. Preparation means and recipes, or modifications thereof, routinely used in directed evolution methods such as traditional PACE methods were utilized certain experiments using robotic PACE.

### General Procedure

The initial phage concentration was 25 μL T7 RNAP M13 phage/500μL LB (425 μL T7 RNAP M13 phage in 8.5 mL LB). The inducer agent was prepared in a separate original reservoir in the staging area. The inducing agent was added using one disposable tip. The liquid was then transferred to the reservoir using clean tips. The “dirty” tips were used for lagoon transfers.

### Set up

At least 30 mL/half hour of culture was available. The chemostat flow-through rate determined the amount of culture available to be pumped on deck during a particular time interval. The flow was set as needed to produce 10 mL/half hour. In various procedures it was set as high as 19 rpm, which would provide 60 mL/half hour. In certain procedures it was set as low as 10 rpm, which provided 30 mL/half hour.

### Amount of Culture Per Operation: 13 mL Reservoir

In the instances when there were 16 wells, 500 μL of culture was prepared for each well resulting in a total of 8 mL of culture. 5 mL of dead volume was provided in the reservoir and 13 mL of bacterial culture was taken to the deck of the system during each operation. The bacterial culture included an accessory plasmid (AP) with the T3 promoter driving pIII.

### Amount of Arabinose Inducer: 325 μL

The procedures were prepared to that the bacterial culture was at a concentration of 25 mM arabinose. Using 1M l-arabinose; 325 μL of the arabinose inducer was added into the reservoir before it was moved to the isolator. Note: the container holding the inducer contained a maximum of 40 mL of inducer, which was at least sufficient to service 123 (or other suitable number of operations), or 2.5 days.

### Plate Reader Plate Use: 4 Plates

A procedure was performed with 14 lagoons. In order to run the procedures for 24 hours, or 48 operations, it was necessary to have 48*14/96=7 plate reader plates. In some procedures that were performed, the number of plates needed was reduced to 4 by down sampling and taking a reading at only odd time points.

### Overview

Before running the PACE procedure using the robotic system various elements of the system were checked. All connections were checked to ensure they were correct, the physical system was reviewed to ensure that no element/component of the system protruded above traverse height. Liquid levels were checked to ensure that the dead volumes entered in software matched those in the physical system. Any not matching were adjusted. The vacuum trap, it was checked and was confirmed to have sufficient room for the procedure. The waste carboy was confirmed to have a capacity greater than the total volume of liquid in the system. The power supply was checked to ensure power supply for isolator was on. The system was checked to ensure that the peristaltic pumps were are on and appropriately connected. The system deck was checked to ensure that the inducer, lagoons that included phage, plate reader plates, and the reservoir were correct and in place for proper action and use during the robotic PACE procedure.

### Robotic Pace Procedure

An overnight culture was started that included 2 mL LB, 200 μL glucose. The temperature of the deck was adjusted to 35° C. The overnight culture was later diluted 1:1000, although the specific dilution was not essential because the chemostat later equilibrated the culture optical density for use. The chemostat was equilibrated to the desired 35° C. temperature, and the deck was set up with the components.

The lagoons were set up with an initial phage concentration of 25 μL T7 RNAP M13 phage/500 μL LB and 300 μL T7 RNAP M13 phage in 6 mL LB; no-phage controls were set up in lagoons in the middle row of the right hand row. The titer of the T7 RNAP M13 page was 10¹¹ pfu/mL. The robotic PACE process was started and 43 iterations were performed (iterations may also be interchangeably referred to herein as “rounds” or “cycles”). Samples removed from receptacles every three hours for assessment. Real-time monitoring was performed in receptacles and/or detection receptacles. Real time monitoring was performed by determining luminescence and/or absorbance of samples.

### Example 2

Evolution of conjugative plasmid conjugation speed is performed using a system of the invention. This procedure includes evolution of conjugative plasmids toward faster conjugation rates, which is accomplished by continuously selecting for conjugative plasmids that are capable of replicating fast enough to stay in a receptacle with an ever-increasing flow-through rate.

### Material and Methods

### Biological Components

(a) F+ bacteria harboring a conjugative plasmid which encodes the sfGFP fluorescent protein.
(b) F+ bacteria not harboring the conjugative plasmid, but that do contain the mutagenesis plasmid MP6 [Badran and Liu (2015) Nature Communications6 (October): 8425.]. Equipment setup (see Example 1 for additional system information and components, also see FIG. 16 which shows an embodiment of the off-deck bacterial chemostat as described below.)

### Off-Ceck Bacterial Chemostat

Including: an incubator, media carboy, waste carboy, peristaltic pumps, and stir plate.

An 80 mL bacterial chemostat is contained within an adjacent 37° C. incubator. It is stirred by a stir plate and magnetic stir bar. Peristaltic pumps exchange liquid in the chemostat for fresh LB media at a flow-through rate appropriate to maintain OD 0.4-0.7, usually 1-3 volumes/hour depending on bacterial strain and preparation. The bacteria cultured in this chemostat are of type (b): they do not harbor the conjugative plasmid.

### Robot Deck Setup:

(i) Bacterial reservoir: a line from the bacterial chemostat, serviced by an arduino-controllable pump, can fetch liquid into an on-deck bacterial reservoir, or drain the reservoir.
(ii) Receptacle plate. A deep-dish 96 well plate harboring 500 μL receptacles. These receptacles contain bacterial culture with a mix of bacteria that contain and do not contain the evolving conjugative plasmid.
(iii) Plate reader plates.
(iv) Tips: 1000 μL tips and 10 μL tips.
(v) Inducer: L-arabinose, the mutagenesis inducer.
(vi) Heaters: appropriate to keep the entirety of the robot deck at 37° C.

### Robot Program:

At each operation the robot performs the following operations:
Fills the on-deck bacterial reservoir with fresh bacterial liquid
Picks up a 10 μL tip. Aspirates L-arabinose. Dispenses L-arabinose into the bacterial reservoir. Disposes of the 10 μL tip.
(Optionally: reads file containing new flow-through rates)
Picks up a 1000 μL tip. Mixes bacterial culture in the bacterial reservoir. Aspirates bacterial culture from the bacterial reservoir. Dispenses bacterial culture into the lagoon wells. Mixes the lagoons. Aspirates waste from the lagoons. Dispenses a 150 μL plate reader sample into a plate reader plate. Disposes of the 1000 μL tips.
Opens the door to the plate reader. Picks up the plate-reader plate, and transfers it to the plate reader. Reads the GFP fluorescence and absorbance of the plate. Returns the plate reader plate to deck.
(Optionally: calls script to analyze plate reader results and update flow-through rates file)
Drains the on-deck bacterial reservoir.
Waits for a timer for the beginning of the next operation.

### Flow-through Rate Details

This evolution works by challenging the conjugative plasmid to replicate and conjugate into new bacteria faster than they are washed out of the lagoon. The appropriate flow-through rate to select for faster conjugation will range from 1-5 volumes/hour. For example, to achieve a flow-through rate of 2 volumes/hour, the robot could perform three operations per hour, exchanging 333 μL of liquid in the lagoon for fresh bacterial culture during each operation.

The flow-through rate can be set to a constant. Alternatively, the (optional) steps above can be implemented to add feedback and control. The plate reader will determine which of the lagoons are demonstrating strong GFP fluorescence, indicating a high concentration of conjugative plasmid. For those lagoons, the flow-rate can be increased. The script will calculate the appropriate new flow-rate and write it to a file, which the robot will read and use to set the number of μL to pipette on the next operation.

These studies utilize an embodiment of a system and method of the invention comprising: a single off-deck chemostat, disposable tips, feedback-control, and addition of inducer.

### Example 3

Experiments using a system for continuous directed evolution that include evolution of T7 RNAP promoter recognition are performed. The procedures include simultaneous evolution of 96 parallel PACE experiments toward recognition of 96 different promoter sequences.

### Biological Components

(i) F+ bacteria containing plasmids encoding pIII.

In each of 96 different plasmids, pIII is under control of a different T7-like promoter. The bacteria additionally harbor the mutagenesis plasmid.
(ii) M13 phage with pIII deleted and replaced with an ORF encoding the T7 RNAP.

### Equipment Setup (see Example 1 for Additional System Information and Components)

### Bacterial chemostat

In this experiment, the bacteria are cultured on-deck. There are 96 different bacteria, each harboring a pIII cassette under control of a different promoter. Each strain is housed in one well of a 24-well plate; there are 4 of these plates total on deck. The 24-well plates are on shakers (shaking is required for chemostats, but not receptacles). An on-deck LB media reservoir, serviced by arduino-callable peristaltic pumps, can be used to draw fresh LB onto the deck.

### Robot Deck Setup

(i) Bacterial reservoir: Four bacterial chemostat plates, each sitting on a shaker plate.
(ii) LB reservoir: serviced by an arduino that controls peristaltic pumps.
(iii) Receptacle plate. A deep-dish 96 well plate harboring 500 μL receptacles. These receptacles contain bacterial culture and phage.
(iv) Plate reader plates. Stacked.
(v) Tips. Four boxes of 1000 μL tips. Four boxes of 10 μL tips. There are two boxes each of “clean” and “dirty” “chemostat” and “receptacle” tips.
(vi) Inducer: L-arabinose, the mutagenesis inducer.
(vii) Heaters: appropriate to keep the entirety of the robot deck at 37° C.

### Robot Program

The individually addressable 8-channel head is used, except where indicated.

At each operation the robot performs the following operations:

### (i) Chemostat Flow-Through

Fills the on-deck LB reservoir with LB
Picks up clean chemostat tips (1000 μL)
Aspirates LB into these tips. Turns of shaking on chemostat containers. Dispenses into the chemostats on-the-fly (without touching).
Re-racks clean chemostat tips (1000 μL).
Picks up dirty chemostat tips (1000 μL)
Mixes chemostat liquid. Aspirates waste. Dispenses to waste.
Reracks dirty chemostat tips (1000 μL)

### (ii) Add Inducer to Chemostat

Picks up clean chemostat tips (10 μL)
Aspirates L-arabinose into these tips. Dispenses into the chemostats on-the-fly (without touching). Turns shaking of chemostats back on.
Re-racks clean chemostat tips (10 μL)
   (iii) Receptacle Flow-Through
Picks up clean receptacle tips (1000 μL) [96-head]
Aspirates bacterial culture from the bacterial chemostat. Dispenses bacterial culture into the receptacles on-the-fly (without touching). [96-head]
Picks up clean receptacle tips (1000 μL) [96-head]
Picks up dirty receptacle tips (1000 μL) [96-head]
Mixes the receptacles'contents. Aspirates waste from the lagoons. Dispenses a 150 μL plate reader sample into a plate reader plate (detection receptacle). Ejects waste. [96-head]
Reracks the Dirty Lagoon Tips (1000 μl) [96-head]
   (iv) Take Measurement of Samples p Opens the door to the plate reader. Picks up the plate-reader plate, and transfers it to the plate reader. Reads the GFP fluorescence and absorbance of the plate. Returns the plate reader plate to deck.

### (v) Clean Up

Drains the on-deck bacterial reservoir.
If plate reader plate is full of samples, use iswap to place it in trash, and take a new plate reader plate from the stack of plate reader plates and place it on deck
Wait for a timer for the beginning of the next operation.

This example demonstrates inclusion use of a system and also inclusion of:
Multiplexed on-deck chemostats, Reused tips, Stacked plate reader plates, Inducer.

### Results

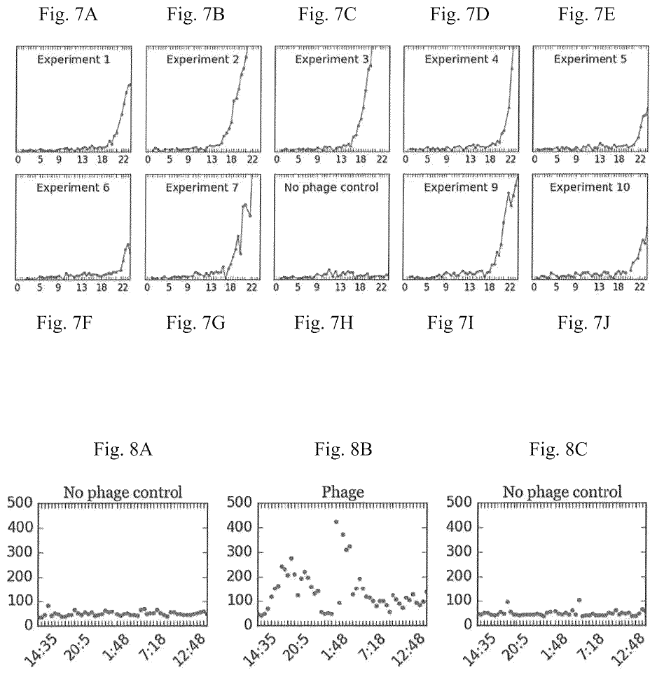

FIG. 7A-J illustrates experimental results for evolving wild type T7 RNAP to bind the T3 promoter. Each graph shows the real time luminescence (corrected for absorbance, in AU), a proxy for the population's activity on the T3 promoter. As shown in FIG. 7A-J, one embodiment of the system for conducting continuous directed evolution disclosed in the present disclosure was used in experiments in which T7 RNAP was evolved to bind the T3 promoter on a liquid handling robot 120, 220 with ten simultaneous phage populations (one of which was a no-phage control experiment). Results indicated no evidence of phage contamination or sub-optimal evolutionary outcomes due to the equipment setup of the system. During this experiment, an on-deck integrated plate reader 160 was used to monitor population luminescence, a proxy for activity on the new T3 promoter, every 30 minutes. Both populations evolved activity on the new promoter, with changes in titer occurring over a timeframe that is equivalent to the same evolution run in a traditional PACE setup. In short, the results showed successful use of a system and methods of the invention to determine luminescence as a measure the current average molecular activity of the evolving phage population in real-time.

### Example 4

See methods and components described in Examples 1-3.

In another continuous directed evolution procedure using a system of the invention, the bacterial reservoir was sterilized by sequential washings with a bleach solution. Cleansing with 10% bleach is a routine, art-known procedure for M13 sterilization, and is used in sterilization of traditional PACE setups. This task may be substantially easier than sterilizing tips that may contain an absorbent filter.

The effectiveness of this cleaning process in a system of the invention was tested. As illustrated in FIG. 8, initial tests showed that this strategy was effective to prevent cross contamination via reused tips. In particular, during the tests, tips were reused and sterilized the on-deck bacterial culture reservoir. FIG. 8A-C illustrates luminescence monitoring for three reservoirs. FIG. 8A and FIG. 8C are graphs depicting the two no-phage control reservoirs, and FIG. 8B shows results of monitoring the reservoir that contained phage. These results, as well as the follow up plaque assays, confirmed that no cross-contamination occurred.

### Example 5

See methods and components described in above Examples, at least, Examples 1-3.

Aspects of the present disclosure are directed to systems and methods for conducting continuous directed evolution (for example PACE procedures) that involve real-time monitoring and feedback control over experimental conditions. Experiments are conducted using systems and methods configured to monitor the status of each independently evolving population in real-time using, for example, a plate reader to measure a detectable parameter, such as luminescence and/or fluorescence produced by engineered reporter constructs in host bacterial cells.

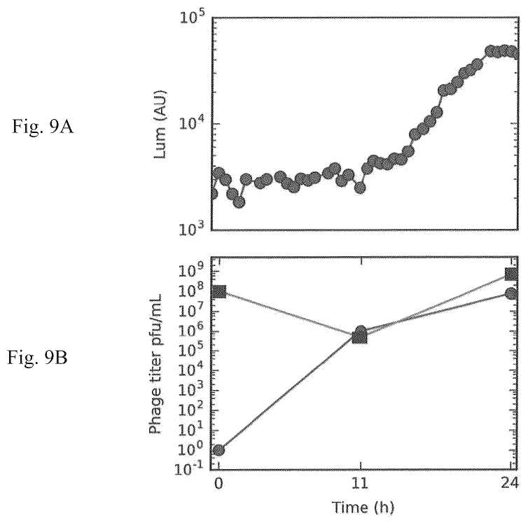

Experiments have been conducted in which luminescence was tied to PACE selection. The methods included production of the limiting phage protein pIII by the host cell, using a proven polycistronic cassette encoding both luciferase and the phage gIII gene. This system was used in experiments to monitor the luminescence of populations in real time, and the results were compared via activity-dependent plaque assays, both of which are proxies for fitness. FIG. 9A-B illustrates results of real-time monitoring in the experiment. FIG. 9A-B provides a comparison between real time readout of luminescence/absorbance (FIG. 9A) and corresponding plaque assays measuring total (triangles) and activity-dependent (circles) phage titer (FIG. 9B).

### Example 6

See methods and components described in above Examples, at least, Examples 1-3.

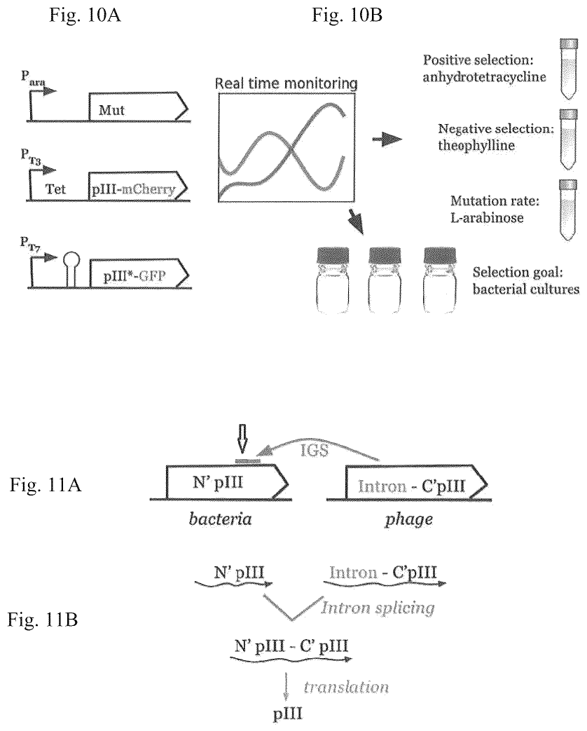

FIG. 10 illustrates an embodiment of a system of the invention used to conduct continuous directed evolution. In the experiment, the system was configured to detect at least one parameter of one or more samples in the receptacles, thereby providing real-time monitoring of the desired and undesired activity of each evolving population. With a feedback controller, the system was configured to respond by making one or more one real-time adjustments in one or more samples based upon the detectable parameter. In some studies, the feedback controller can be configured to adjust the mutation rate, the positive and negative selection strengths, and/or the selection goal in a programmable manner.

More specifically, FIG. 10 illustrates results of feedback and control using the system for conducting continuous directed evolution, such as PACE. FIG. 10A illustrates that the T7 RNAP promoter recognition selection involves two readouts: mCherry for positive selection and GFP for negative selection. Three small molecules control it: L-arabinose controls mutagenesis, anhydrotetracycline modulates positive selection, and a theophylline-sensitive riboswitch controls negative selection. FIG. 10B illustrates that real-time monitoring of the two readouts can be incorporated into robotic control over all three chemical inducers, as well as providing different bacterial cultures.

Using such a configuration, a system of the invention is used for continuous directed evolution procedures, such as PACE, in which control over selection stringency is demonstrated by successfully performing parallel PACE experiments with the drift plasmid and theophylline-titratable negative selection in which induction of strong negative selection early on leads to washout, drift induction leads to indefinite propagation, and inducing neither produces results equivalent to the absence of both inducer systems.

### Example 7

See methods and components described in above Examples, at least, Examples 1-3.

Various configurations and components are utilized in continuous directed evolution using systems and methods of the invention.

In an experiment, a continuous directed evolution procedure is performed in a system comprising on-deck sterilizing and re-use of components, which assists in performing massively parallel directed evolution. To prevent cross-contamination, each receptacle is assigned a tip that is used for all of its liquid transfer operations. Thus, this tip is always contaminated with phage specific to its receptacle and need not be sterilized. These dirty tips may aspirate fresh bacterial culture, dispense it into their respective receptacles, mix, and then may be stored in special 96-well tip holders with dividers to prevent them from touching during pick-up and drop-off. In this system, it may be desirable to then clean the fresh bacterial culture reservoir, each well of which has been contaminated by a dirty tip.

In an experiment, a continuous directed evolution procedure is performed using a system of the invention in order to conserve deck space a plurality of well-plates is stacked on top of each other. In this procedure the liquid handling robot is configured to selectively un-stack the plates so that the robot can access a particular set of samples and perform the steps for the continuous directed evolution process.

In an experiment, a continuous directed evolution procedure is performed using a system of the invention, tips are re-used and sterilized to prevent cross contamination. In this procedure the system is be configured for automatically bleach-sterilizing on-deck parts, including tips. In certain procedures the system is configured to have a separate set of tips for each liquid exchange fluid used during the liquid exchange process. For example, the procedure includes a separate set of 96 tips for 1) the host sample cells; 2) each chemical inducer; 3) one set for the integrated plate reader 160; and 4) one for each lagoon/plate, which allows for up to four plates of evolving populations to be run in parallel.

In an experiment, a continuous directed evolution procedure is performed using a system of the invention, and the system and methods used to conduct the continuous directed evolution, (for example PACE) are configured to extend real-time monitoring to collect data on how strongly each population is triggering both positive and negative selection. Negative selection during PACE may involve making unwanted activity result in production of a dominant negative variant of pIII, called pIII*, that competes with functional pIII for space on the tail of progeny phage. The range of acceptable concentrations of pIII* is very narrow, and thus negative selections often require extensive tuning.

In an experiment, a continuous directed evolution procedure is performed using a system of the invention that integrates a mCherry in a polycistronic cassette with pIII and GFP with pIII*, allowing an integrated plate reader to monitor desired and undesired molecular activities for each population.

In an experiment, a continuous directed evolution procedure is performed using a system of the invention that is configured to minimize time required for measurement, by not using both fluorescence and luminescence measurements.

In an experiment, a continuous directed evolution procedure is performed using a system of the invention in which the system is configured to permit simultaneous measurement of pIII and pIII* transcription over 24 continuous hours without interruption during a Robotic PACE evolution experiment.

In an experiment, a continuous directed evolution procedure is performed using a system of the invention that is configured to complete the feedback loop by enabling the system to change the selection stringency and selection goal in response to the real-time monitoring data. With the system, the positive selection strength is titratable using a plasmid that produces pIII from a separate copy of gIII in response to anhydrotetracycline. Induction relaxes selection by producing “free” pIII, with high levels resulting in effective genetic drift. The mutation rate is inducible with arabinose. In certain experiments in this system, the negative selection strength is titratable by incorporating a theophylline-inducible riboswitch.

In an experiment, a continuous directed evolution procedure is performed using a system of the invention is used to replicate aspects of prior conventional PACE experiments, except that the system is configured to run at least 384 parallel experiments with real-time monitoring and feedback control to identify appropriate feedback responses to population activities. Whereas previous studies investigated combinations of either a fixed ‘high’ or ‘low’ amount of inducer to control mutation rate and selection stringency, the systems and methods of the invention for conducting continuous directed evolution, such as PACE, as disclosed herein is capable of more comprehensively investigating outcomes using finer inducer concentration resolution, and/or periodic schedules.

In an experiment, a continuous directed evolution procedure is performed using a system of the invention that is configured to test the effects of sinusoidally oscillating the mutation rate with a period of five hours, and quantify the benefits of periodically switching the selection goal - an experimental test of the hypothesis that varying the evolutionary environment may speed up evolution.

In an experiment, a continuous directed evolution procedure is performed using a system of the invention configured to demonstrate how the mCherry and GFP fluorescence readouts of activity can be used to modulate the amount of theophylline added to the phage population in order to hold the negative selection at optimal strength even while the fitness of each population changes.

The experiments described in Example 7 are provided to demonstrate different embodiments of systems and methods of the invention. It will be understood that two more of the embodiments of the system and methods that are illustrated in the experiments set forth in Example 7 can be combined in a single system as appropriate.

### Example 8

See methods and components described in above Examples, at least, Examples 1-3.

Studies are performed with embodiments of systems and methods of the invention to evolve introns to trans-splice and edit target RNAs. A goal is to develop a selection for trans-splicing introns. The ability for RNA editing solves several longstanding problems afflicting PACE systems while also empowering other biomedical researchers.

### There Are Several Mechanisms by Which Rna Editing Enhances Pace:

### Keeping Part of a Gene Constant During Evolution:

Trans-splicing between an evolving phage-encoded gene fragment and a static bacteria-encoded fragments allows researchers to selectively evolve only part of a gene, thereby preserving the interaction between a component that must remain constant for the target application but cannot be selected for in the context of PACE.

### Liberating Pace From the Tyranny of the Small Replicator:

Because small genomes replicate more quickly, PACE phages encoding large biomolecules suffer a fitness penalty. This distorted selection pressure can dominate performance of a variant, especially at the beginning of an experiment when activity is poor or when selection is relaxed to escape a local fitness peak trap. It is a longstanding technical problem that phage will excise the gene of interest rather than evolve improved activity. Introns can reduce the size penalty and eliminate this problem by splitting genes for which the entire gene need not evolve, such as when a small evolving domain is fused to a larger constant protein such as dCas9.

### Resistance to Cheaters:

Splitting the bacteria-encoded selection machinery into multiple pieces prevents a single recombination event from creating a “cheater” phage that can produce its own pIII and evade the selection. This problem has previously derailed several traditional PACE experiments.

Multiple Simultaneous Positive and/or Negative Selections and Contexts:

In the past, there have been numerous occasions in which it would be advantageous to select in favor of multiple properties simultaneously: for example, activity on a new substrate and solubility. Previous PACE strategies, in which both desired properties are selected for sequentially or simultaneously, have proven to be unreliable because they tend to evolve cheater proteins that strongly satisfy just one of the properties. Trans-splicing introns provide an elegant solution to this longstanding technical problem: each desired activity can be linked to production of a portion of pIII. Only when all of the activities are satisfied will the full gene be assembled via intron splicing.

Intron evolution enhances traditional PACE utility, and is a method with which to evolve introns to edit any RNA that has many future therapeutic and research applications such as in (1) gene therapy, in which in vivo RNA editing can correct genetic diseases by repairing faulty RNAs produced by genes for which CRISPR genome editing would be too difficult or hazardous, such as Huntington's or Bcr-Abl; (2) genetic safeguards, in which engineered genes split into multiple pieces are exponentially less likely to be transmitted intact to wild organisms; and (3) precise expression control in which RNA editing permits genetic activity to be restricted to a narrow developmental window or tissue type in which both halves of a separately encoded split gene are transcribed. Customized introns trans-splice into and thereby precisely edit any target RNA both swiftly and reliably.

Experiments are performed and a high-efficiency trans-splicing intron is created. The process is a multi-step procedure to produce a high-efficiency trans-splicing intron for a target gene relevant to PACE. Steps include: applying an existing computational technique to predict effective splice sites and design intron sequences that will target these sites (Meluzzi et al. (2012) RNA. March; 18(3): 590-602. Robotic PACE systems and methods of the invention are used to evolve several of these predicted introns in parallel, and the efficiency and speed of each intron is optimized to its specific context. The computational model is adapted to better predict effective introns using the resulting data.

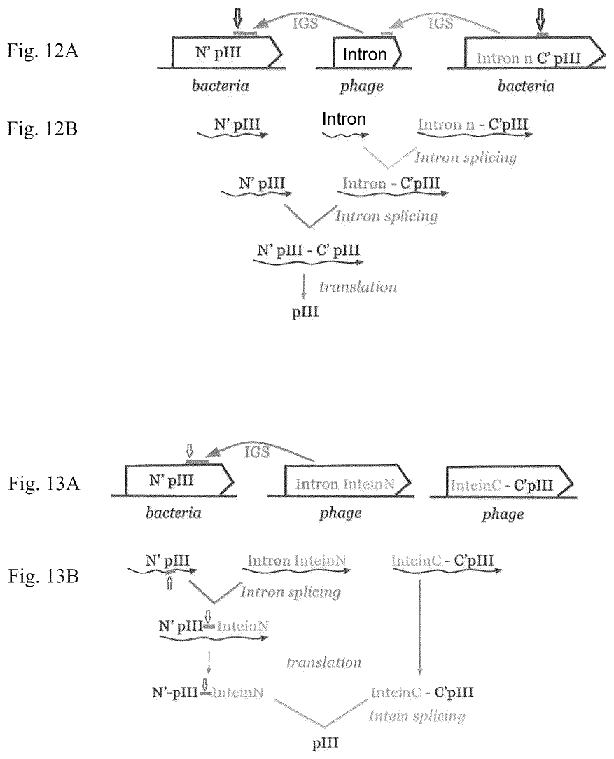

Experiments to evolve a trans-splicing intron to efficiently splice into a desired target sequence, begin with the Tetrahymena group I intron, which has little preference for the sequence of their attached 3′ exons and can be directed to trans-splice into a 5′ exon directly via an Internal Guide Sequence (IGS) (Zettler, Joachim et al., FEBS letters 583.5 (2009): 909-914; Müller, Ulrich F. Molecules 22.1 (2017): 75). The selection splits the critical phage gene III, the basis for PACE selection, just after the signal sequence. Previous phage display experiments have shown that this location tolerates arbitrary amino acid insertions. The evolving phage genome encodes the intron, the desired 3′ exon, and the 3′ portion of gene III (illustrated in FIG. 11A-B). The 5′ piece and target splice site is encoded in the host bacteria and therefore remains constant. Correct splicing generates an RNA encoding functional pIII. After signal peptide processing, the resulting protein has an extra N-terminal sequence just after the splice site corresponding to the unique 5′ and 3′ exons of the target splice site. At least one group I intron is evolved that is capable of supporting PACE propagation by reconstituting a split gene III. A variety of other target DNA sequences are inserted between the gene III signal sequence and the rest of the gene and at least nine additional introns are evolved that are capable of supporting PACE propagation by splicing into those sequences. Target sequences include different sequences within gene III and T7 RNA polymerase as well as two inteins.

Systems and methods of the invention for robotic PACE are used to collect a large data set relating splice sites to efficient corresponding intron sequences and the data is used to optimize computational intron design. The evolutionary trace method (Lichtarge, Olivier, et al., (1996) Journal of Molecular Biology 257(2) : 342-358), which is traditionally used to infer which residues of a protein confer functionality given phylogenetic data, is adapted to analyze which mutations in the intron determine its exon sensitivity given sequencing data from evolved PACE variants. Experimental results permit improvement in the predictive algorithm that identifies splice sites and corresponding introns.

Additional experiments are performed and generate a second context for selection by splitting the T7 RNA polymerase gene at a sequence corresponding to a solvent-exposed loop known to tolerate splitting and insertions (Pu, Zinkus-Boltz, & Dickinson, B.C. 2017 Nature Chemical Biology 13(4): 432-438). Its reconstitution is selected by splicing and the subsequent transcription of gene III. A single properly spliced RNAP generates many copies of pIII, meaning this version of the selection is capable of evolving very low-efficiency introns.

If introns evolved for RNA editing are too promiscuous outside of the context of phage, a selection against unwanted 5′ splice sites is developed by engineering the host cell to produce a large excess of RNAs carrying these sequences and the selection stringency is tuned for the correct splice site as needed using feedback control. If group I introns have a stronger preference for a specific 3′ exon context than is previously expected, results show that the 3′ exon sequence encoded on the phage to consistently evolves. The intron is forced to adapt to a fixed 3′ exon by utilizing a second intron in the selection. This second intron is placed inside of the evolving intron, near its 3′ end, and trans-splices the 3′ side of the pIII transcript (FIG. 12A-B). This second trans-splicing allows both halves of pIII, and consequently both exon sequences, to be encoded in the bacterial genome.

### Example 9

See methods and components described above Examples, at least, Examples 1-3.

Embodiments of methods and systems of the invention include robotic PACE post-translational optogenetic control over protein activity. In these experiments, ultrafast light-based control of protein splicing is developed that permits quick modulation of PACE experimental conditions. Prior methods do not permit rapid and precise control of the activity of arbitrary proteins within engineered cells. Some embodiments of robotic PACE systems and methods of the invention as described in examples above herein utilized inducible promoters to control the mutation rate and positive selection stringency. Additional experiments are performed using optogenetic intein splicing to control protein activity, which permits finer temporal control by bypassing transcription, translation, and possibly much of protein folding.

Inteins can become nonfunctional, or suffer an efficiency penalty, when placed in a new context. To adapt a given intein to function in its new context, procedures include surrounding it with the desired extein sequence and selecting in favor of variants that splice. These exteins are inserted just after the signal peptide in pIII, or alternatively into the H-loop of T7 RNA polymerase (Shis, David L., & Bennett, M.R., (2013) PNAS USA 110(13): 5028-5033; Pu, Zinkus-Boltz, & Dickinson, B.C. 2017 Nature Chemical Biology 13(4): 432-438), because both locations allow insertion of arbitrary extein sequences without interfering with the protein's function. To keep the exteins constant while evolving both halves of the intein, the former is encoded in the host bacteria and the latter on the phage (FIG. 13A), then they are spliced together using introns generated as in Example 8. Because RNA splicing necessarily occurs before protein splicing, the bacteria-encoded RNA transcripts encoding the exteins are first be spliced together with the evolving phage-encoded intein transcripts. This places the fixed, non-native extein sequence adjacent to the intein. When both are translated, the two splice together to create an immediately functional protein (FIG. 13B).

Further studies are performed and feedback control in Robotic PACE is improved by accelerating fluorescent protein maturation time through evolving an intein that splices together two already-folded pieces. Additional studies are carried and an intein is evolved that splices together fragments of pIII/pIII*. In addition evolved inteins are rendered sensitive to light by fusing an optogenetic domain onto the intein. This strategy relies on optogenetic proteins that undergo a conformational change in response to light; the optogenetic protein sterically interferes with protein splicing in one conformation but not the other, placing the intein under optogenetic control. There are numerous examples of this process in the literature, involving a variety of optogenetic proteins and inteins (Wong, Stanley, et al., PloS one 10.8 (2015): e0135965; Ren, Wei, et al., J. Am. Chem. Soc. 137.6(2015): 2155-2158; and Tyszkiewicz, Amy B., & Muir, T.W. (2008) Nature Methods 5(4): 303-305); in conjunction with embodiments of systems and methods of the invention. Additional studies are performed and pIII and pIII* intein splicing is rendered light-sensitive thus enabling optogenetic control of selection stringency.

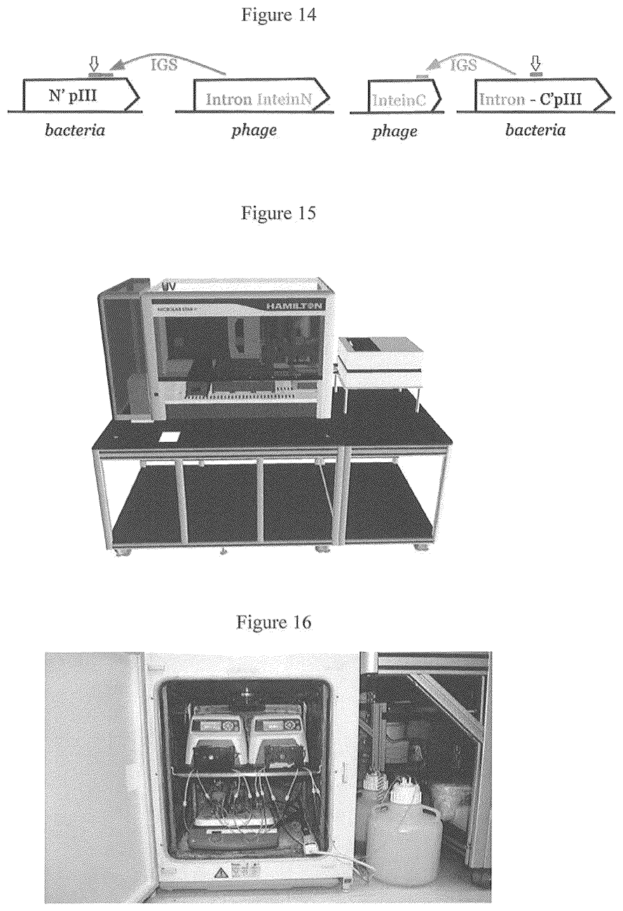

An additional study is performed and pIII and pIII* intein splicing is rendered light-sensitive and enables optogenetic control. In some experiments, the entire protein to be spliced is inserted within the highly tolerant solvent-exposed H-loop of &7 RNA polymerase, a site that permits splitting, fusions and inducible reassembly (see: for example Pu, Zinkus-Boltz, & Dickinson, B.C. 2017 Nature Chemical Biology 13(4): 432-438). In addition faster splicing variants are evolved and used in the selection circuit. To evolve extein non-sensitivity on both sides simultaneously, procedures are performed implementing a two-intron selection (see FIG. 14) and splicing times are further optimized.

### Example 10 Multiplexed T7→T3 Evolution

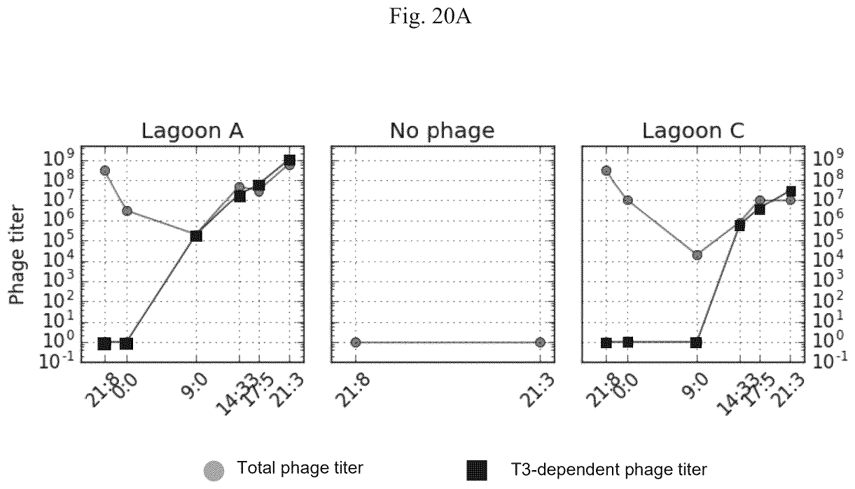

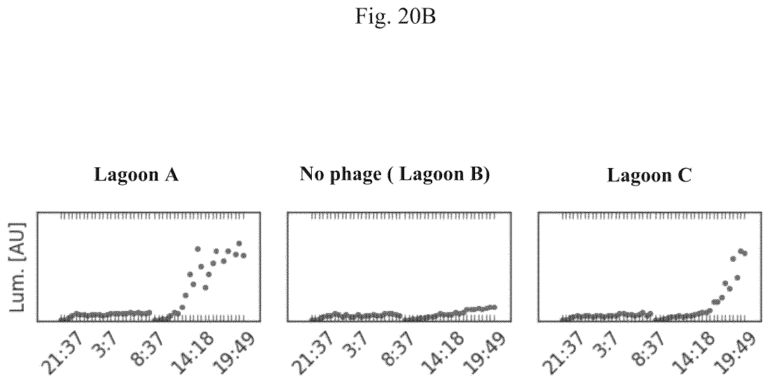

In this embodiment, a liquid handling robot was used to perform three simultaneous experiments designed to replicate a previously published evolution in which the T7 RNA polymerase was evolved to recognize the T3 promoter (Esvelt, Kevin M. et al. 2011. Nature 472 (7344):499-503). Three simultaneous experiments were performed at a rate of 1 volume per hour (vol/hr): two evolutions (lagoons A and C) and one no-phage control (lagoon B). Luminescence, a proxy for activity on the T3 promoter, was monitored using an on-deck plate reader every 30 minutes. Plaque assays for total phage and T3-activity dependent plaques were performed to confirm luminescence results. Bacteria in both lagoons A and C evolved activity on the T3 promoter. FIG. 20A-B illustrates results of experiments that included multiplexed T7→T3 evolution. An embodiment of a system and liquid handling robot was used in these experiments to conduct continuous directed evolution.

### Materials and Methods

### Experimental Setup

The deck of the liquid handling robot was heated to 37° C. A 300 mL turbidostat containing bacteria was held at OD=0.5 and cultured at 37° C. Bacteria from the turbidostat were pumped into an on-deck reservoir. Optionally, in some embodiments the reservoir was placed on a shaker plate on the deck of the liquid handling robot.

Three independent experiments of 500 μL each were housed in a deep-dish 96-well plate in alternating rows. The procedure used is similar to that described in Example 11, except that in the small-scale experiment described in Example 10, the on-deck bacterial reservoir did not need to be shaken because its size was sufficiently small so the inducer diffused without agitation, making the shaker optional.

### Robot Program

At each operation the robot performed the following operations:
Filled the on-deck bacterial reservoir with fresh bacterial liquid from the turbidostat.
Picked up a 300μL tip. Aspirated L-arabinose. Dispensed L-arabinose into the bacterial reservoir to 10 mM to induce mutagenesis. Disposed of the 300 μL tip.
Picked up a 1 mL tip. Mixed the induced bacterial culture in the bacterial reservoir. Aspirated 250 μL of bacterial culture from the bacterial reservoir and dispensed bacterial culture into lagoon wells. Mixed the lagoons. Aspirated 250 μL waste from the lagoons.
(Optionally: in some experiments, the robot dispensed 175 μL into a plate reader plate.)
Dispensed into waste and disposed of the 1 mL tips.
(Optionally: in some experiments the robot used the gripper arm to transport the plate reader plate to the integrated plate reader, read it, and then return it to deck.) In some experiments the plate reader was a ClarioSTAR plate reader.

In some experiments, the robot implemented the foregoing program once every half hour, resulting in a 1 vol/hr turnover in liquid in each experiment. Certain equipment and components used in the experiments are described elsewhere herein, for example, see Examples 1-3.

### Results

Real-time luminescence monitoring results (FIG. 20B) were compared with plaque assays (FIG. 20A) for total phage (circles) and T3-activity dependent plaques (squares). As the population became able to propagate on the evolutionary goal, the total phage titer dropped and then recovered (FIG. 20A, left and right panels). In the no-phage control experiment (FIG. 20A, middle panel), no phage were observed for the duration of the experiment, confirming that cross-contamination did not occur. Phage capable of forming plaques in a T3-activity-dependent plaque assay arose by 12 hours of continuous flow and overtook the population by 24 hours. Both plaque-assay metrics showed T3 activity, confirming the luminescence data. All three analysis methods showed lagoon A developing T3 activity before lagoon C. The results confirmed that luminescence tracked the activity dependent plaque assay results.

The experiments that were performed confirmed that embodiments of methods and systems of the invention can be used for parallel multiplexed continuous directed evolution in several samples (lagoons). In one instance, the methods permitted simultaneous continuous directed evolution in 3 lagoons, with real-time adjustment of conditions possible using the system. Absorbance and luminescence were used as detectable parameters with which to identify characteristics of the samples. In some of the experiments, absorbance and luminescence were used as indicators of infectious phage activity, and were monitored using a plate reader at time intervals. Monitoring absorbance and luminescence permitted determination of characteristics of the samples in receptacles in the system, and permitted real-time adjustments in one or more of the receptacles based on the identified characteristics of the samples in the receptacles.

### Example 11 Massively Multiplexed tRNA Evolution

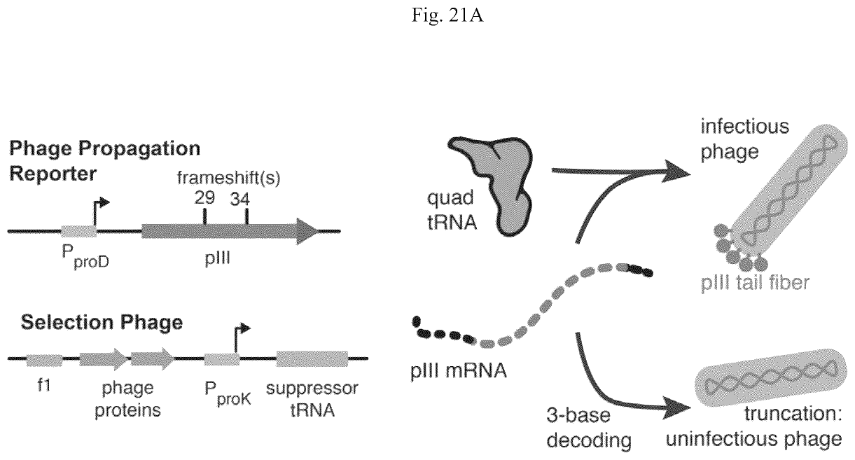

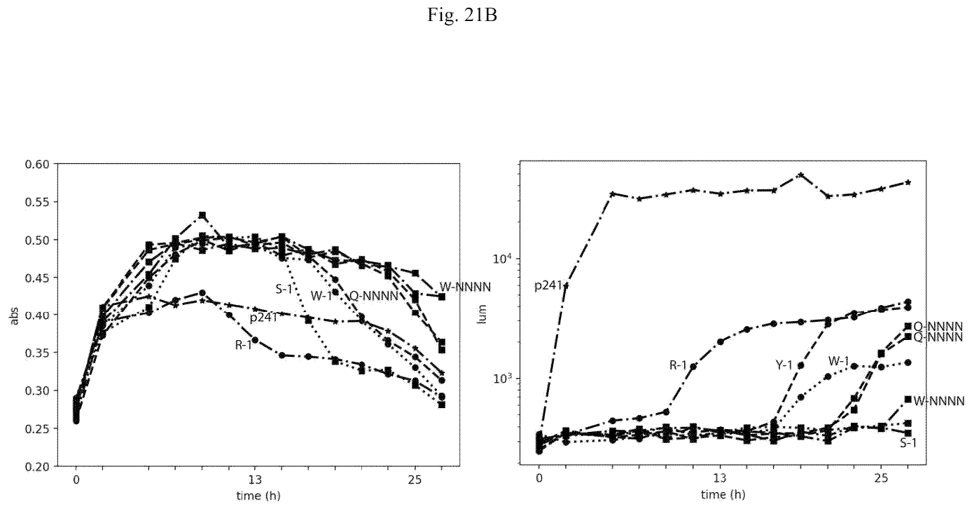

Experiments were performed using an embodiment of a system of the invention to conduct parallel multiplexed continuous directed evolution in 48 lagoons. In this embodiment, a liquid handling robot was used to perform 48 simultaneous experiments using tRNAs that were previously evolved for improved ability to decode quadruplet codons using traditional continuous evolution setups. In this embodiment, a tRNA was encoded on an M13 bacteriophage, from which pIII had been deleted. These bacteriophage were challenged to propagate in E. coli bearing a plasmid that contained a copy of pIII which had been encoded using a quadruplet codon. A mutagenesis plasmid, MP6 (Badran, Ahmed H. & Liu, D. R., 2015. Nature Communications 6 (October):8425), was also used to elevate the mutation rate. Absorbance and luminescence, a proxy for infectious phage activity, were monitored using the on-deck plate reader every 30 minutes. FIG. 21A-B illustrates results from use of an embodiment of a system of the invention to conduct multiplexed continuous directed evolution of a large number of samples.

### Materials and Methods

### Constructs and Experimental Design

The phage propagation reporter plasmid encoded pIII, an essential phage protein, which had been encoded with one or more quadruplet codons at permissive residue(s) 29 or 34. The quadruplet-decoding tRNA was encoded on an M13 phage from which pIII had been deleted.

When challenged to infect E. coli-bearing the phage propagation reporter plasmid, the Selection Phage entered the cell. Successful decoding of the quadruplet codon resulted in translation of full-length pIII and, thereby, infectious phage progeny. A failure to decode the quadruplet codon resulted in a frameshift, premature termination, and a truncated, non-functional pIII. Plate reader measurements were used to track the absorbance and luminescence of the experiments as they progressed.

### Experimental Setup

The deck of the liquid handling robot was heated to 37° C. A 300 mL turbidostat containing bacteria was held at OD=0.5 and cultured at 37° C. Bacteria from the turbidostat were pumped into an on-deck reservoir. The reservoir was on a shaker plate on the deck of the liquid handling robot.

Forty-eight independent experiments of 500 μL each were housed in a deep-dish 96-well plate in alternating rows. The procedure used was similar to that described in Example 10, except in the larger-scale experiment described in Example 11, the on-deck bacterial reservoir was agitated on a shaker to assist in diffusion of the inducer.

### Robot Program

At each operation the robot performed the following operations:
Filled the on-deck bacterial reservoir with fresh bacterial liquid from the turbidostat. The on-deck bacterial reservoir was on a shaker plate.
Picked up a 300 μL tip. Aspirated L-arabinose. Dispensed L-arabinose into the bacterial reservoir to 10 mM to induce mutagenesis. Shook the shaker plate to mix. Disposed of the 300 μL tip.
Picked up a 1 mL tip. Mixed the induced bacterial culture in the bacterial reservoir. Aspirated 250 μL of bacterial culture from the bacterial reservoir and dispensed bacterial culture into lagoon wells. Mixed the lagoons. Aspirated 250 μL waste from the lagoons.
(Optionally: in some experiments the robot dispensed 175 μL into a plate reader plate.)
Dispensed into waste and disposed of the 1 mL tips.
(Optionally: in some experiments the robot used the gripper arm to transport the plate reader plate to the integrated plate reader, read it, and then return it to deck.) In some experiments the plate reader was a ClarioSTAR plate reader.

In some experiments, the robot implemented the foregoing program once every half hour, resulting in a 1 vol/hr turnover in liquid in each experiment. Certain equipment and components used in the experiments are described elsewhere herein, for example, see Examples 1-3.

### Results

Plate reader absorbance and luminescence measurements were used to track the real-time progress of the experiments. Lagoons with high phage titer showed depressed luminescence, allowing identification of high-activity lagoons as well as lagoons that evolved recombinants. The previously evolved p241 bacteriophage immediately triggered activity-dependent luminescence monitoring (FIG. 21B, right panel), confirming that it was already evolved to perform well on this evolution goal. All other lagoons required evolution in order to identify an evolution solution, which arose by 26 hours after the experiment began.

Prior experiments using traditional methods showed that R-, Y-, W-, and Q-tRNA scaffolds had successfully evolved improved variants. Robotic continuous evolution methods were used with these and other scaffolds and the results confirmed the success and efficacy of the robotic methods. FIG. 21 A-B illustrate certain of the results. The clonal phage sequenced from the R-, Y-, W-, and Q-experiments revealed confirmatory known variants, as well as new variants.

Absorbance was depressed for experiments with high phage titer, including all experiments that triggered luminescence monitoring, as well as ESP1xS-1, which was later confirmed to have evolved a recombinant phage. Plaque assays of the final experiment liquid confirmed that the “no-phage control” experiments remained uncontaminated at the end of the 27-hour experiment.

The experiments performed confirmed that embodiments of methods and systems of the invention can be used for parallel multiplexed continuous directed evolution in high numbers of samples (lagoons). In one instance, the methods permitted simultaneous continuous directed evolution in 48 lagoons, with real-time adjustment of conditions possible using the system. Absorbance and luminescence were detectable parameters used to identify characteristics of the samples. Absorbance and luminescence were used as indicators of infectious phage activity, and were monitored using a plate reader at time intervals. The monitoring permitted determination of characteristics of the samples in receptacles in the system, and the option of making real-time adjustments in one or more of the receptacles based on the identified characteristics of the samples in the receptacles.

Collectively, systems, methods, and kits of the invention for conducting robotic continuous directed evolution according to the present disclosure constitute the first feedback and control system for an evolving population, and are capable of adjusting every major parameter thought to be relevant to evolutionary success. The foregoing detailed description and examples below have been presented for the purposes of illustration and description.

They are not intended to be exhaustive or to limit the invention to the particular disclosed embodiments. Numerous variations and configurations will be apparent in light of this disclosure.

### Equivalents

It is to be understood that the methods and compositions that have been described above are merely illustrative applications of the principles of the invention. Numerous modifications may be made by those skilled in the art without departing from the scope of the invention.

Although the invention has been described in detail for the purpose of illustration, it is understood that such detail is solely for that purpose and variations can be made by those skilled in the art without departing from the spirit and scope of the invention which is defined by the following claims.

The contents of all literature references, publications, patents, and published patent applications cited throughout this application are incorporated herein by reference in their entirety.

1-24. (canceled)

25. A method for conducting discretized continuous directed evolution simultaneously in a plurality of samples, the method comprising:
providing one or more receptacle in a plurality of receptacles, with one or more samples, wherein (i) each sample is in a separate receptacle in the plurality of receptacles; (ii) one or more independently selected receptacles in the plurality of receptacles comprises a cell; and (iii) one or more independently selected receptacles in the plurality of receptacles comprises one or more horizontally transferable nucleic acids;
providing a liquid handling robot configured to perform liquid exchange in one or more receptacles in the plurality of receptacles;
facilitating mixing in one or more receptacles in the plurality of receptacles;
detecting at least a first detectable parameter in one or more of the samples in the plurality of samples; and
modifying one or more of the samples in the plurality of receptacles in real-time based at least in part on the first detectable parameter of the one or more of the samples.

26. The method of claim 25, wherein at least one of the horizontally transferable nucleic acids is undergoing directed evolution.

27. The method of claim 25, wherein one or more cells in the one or more samples does not comprise a horizontally transferable nucleic acid.

28. The method of claim 27, wherein at least one of the one or more cells not comprising the horizontally transferable nucleic acid is undergoing directed evolution.

29. The method of claim 25, wherein the mixing is within the sample.

30. The method of claim 29, wherein the facilitated mixing comprises mixing (a) one or more of the cells not comprising a horizontally transferable nucleic acid undergoing directed evolution and (b) one or more of the cells comprising a horizontally transferable nucleic acid undergoing directed evolution.

31. The method of claim 25, wherein the horizontally transferable nucleic acids are secreted by at least a portion of the cells in the plurality of receptacles, wherein the means of secreting comprises a DNA export system.

32. The method of claim 25, wherein the horizontally transferable nucleic acids encode one or more genes that produce one or more biomolecules that exhibit at least one direct sensor-detectable parameter.

33. The method of claim 25, wherein one or more of the cells produce an indirect sensor-detectable parameter when triggered by the production of one or more biomolecules encoded by the horizontally transferable nucleic acids.

34. The method of claim 25, wherein a feedback controller is also provided and is configured to make at least one real-time adjustment in one or more of the samples in the plurality of receptacles based upon the first detectable parameter.

35. (canceled)

36. The method of claim 25, wherein the detection of the first detectable parameter determines a characteristic of one or more of the samples in the plurality of receptacles, wherein the determined characteristic comprises at least one of: selection, positive selection, negative selection, an enzyme activity, phage population size, bacterial population size, gene transcription, and biomolecule evolution in one or more of the samples in the plurality of receptacles.

37. The method of claim 25, wherein one or more of the samples in the plurality of receptacles comprise a population of bacteriophage.

38-39. (canceled)

40. The method of claim 25, further comprising, determining a characteristic of one or more of the samples, based at least in part on the first detectable parameter, wherein the determined characteristic comprises at least one of:
selection, positive selection, negative selection, an enzyme activity, phage population size, gene transcription, biomolecule evolution, and promoter activity in the one or more of the samples.

41. (canceled)

42. The method of claim 25, wherein:
the first detectable parameter comprises one of more of absorbance, luminescence, and fluorescence; and
one or more samples in the plurality of samples are modified in real-time based upon at least one of the detected absorbance, luminescence, and fluorescence.

43. (canceled)

44. The method of claim 25, wherein one or more of the samples comprise a bacteriophage population, and wherein the modification of one or more samples in the plurality of receptacles is selected for one or more of:
(a) modifying a positive selection strength of at least one sample comprising a bacteriophage population in response to the first detectable parameter;
(b) modifying a negative selection strength of at least one sample comprising a bacteriophage population in response to the first detectable parameter;
(c) modifying a mutation rate of at least one sample comprising a bacteriophage population in response to the first detectable parameter; and
(d) modifying a selection goal of at least one sample comprising a bacteriophage population in response to the first detectable parameter.

45. The method of claim 25, wherein modifying one or more of the samples in the plurality of receptacles comprises adding an inducing agent to one or more of the samples in the plurality of receptacles in response to the first detectable parameter.

46. The method of claim 25, further comprising:
detecting a second parameter in one or more of the samples in the plurality of receptacles; and
modifying one or more samples in the plurality of receptacles in real-time based at least in part on the second detected parameter of the one or more of the samples.

47. The method of claim 25, further comprising:
providing one or more reservoirs, wherein each reservoir is configured to hold and selectively dispense an independently selected liquid exchange fluid;
sterilizing one or more of the reservoirs with a solution comprising bleach; and
placing one or more of the independently selected liquid exchange fluids into a selected reservoir of the one or more reservoirs, wherein the one or more independently selected liquid exchange fluids comprise one or more of a bacterial culture, a bacteriophage solution, and an inducing agent.

48-50. (canceled)

51. The method of claim 47, wherein the sterilizing step comprises:
washing the reservoir with a bleach solution comprising at least 8%, 9%, 10%, 11%, or 12% bleach; and rinsing the reservoir with a solution comprising water.

52-53. (canceled)

54. The method of claim 25, wherein the detecting step comprises transferring an aliquot of one of the samples from the receptacle containing the sample into a detection receptacle, and detecting the at least one parameter in the aliquoted sample while the aliquot is in the detection receptacle.

55-110. (canceled)
